(19) United States
(12) Patent Application Publication
(10) Pub. No.: US 2024/0225502 A1
(43) Pub. Date: Jul. 11, 2024

(54) PLASMA COLLECTION BASED ON DONOR EXTRACELLULAR FLUID

(71) Applicant: FENWAL, INC., LAKE ZURICH, IL (US)

(72) Inventors: Amit J. Patel, Algonquin, IL (US); Samantha M. Planas, Wauconda, IL (US); Walter T. Watts, Lake Forest, IL (US); Kyungyoon Min, Kildeer, IL (US); Daniel R. Boggs, Libertyville, IL (US); Thomas J. Gniadek, Deerfield, IL (US)

(21) Appl. No.: 18/584,182

(22) Filed: Feb. 22, 2024

Related U.S. Application Data
(63) Continuation of application No. PCT/US2022/027484, filed on May 3, 2022.
(63) Continuation-in-part of application No. 18/371,795, filed on Sep. 22, 2023.
Application No. 18/371,795 is a continuation of application No. 17/861,437, filed on Jul. 11, 2022, now Pat. No. 11,801,001.
Application No. 17/861,437 is a continuation of application No. 17/306,099, filed on May 3, 2021, now Pat. No. 11,412,967.
Application No. 17/306,099 is a continuation-in-part of application No. 16/739,441, filed on Jan. 10, 2020, now Pat. No. 11,383,013.
Application No. 16/739,441 is a continuation of application No. PCT/US2019/033318, filed on May 21, 2019.
(60) Provisional application No. 63/256,762, filed on Oct. 18, 2021.
(60) Provisional application No. 63/244,321, filed on Sep. 15, 2021.
(60) Provisional application No. 63/236,743, filed on Aug. 25, 2021.
(60) Provisional application No. 63/140,534, filed on Jan. 22, 2021.
(60) Provisional application No. 62/846,400, filed on May 10, 2019.
(60) Provisional application No. 62/752,480, filed on Oct. 30, 2018.
(60) Provisional application No. 62/674,144, filed on May 21, 2018.

(51) Int. Cl. A61B 5/15

(52) U.S. Cl. CPC A61B 5/150992; A61B 5/150229; A61B 5/150251; A61B 5/150343; A61B 2560/0487

(57) ABSTRACT

A system and method for collecting plasma comprises a control circuit configured to control operation of the system, the control circuit configured to receive one or more donor parameters. The control circuit estimates a physiological fluid amount of the donor based at least in part on the one or more donor parameters and calculates a target amount of plasma product comprising raw plasma and anticoagulant by multiplying a prestored constant by the estimated physiological fluid amount. The control circuit controls the system to operate draw and return phases to process whole blood into plasma product until a measured amount of plasma product in the collection container meets the target amount of plasma product.

### CROSS-REFERENCE TO RELATED APPLICATIONS

This application is a continuation of International Application No. PCT/US2022/027484, filed May 3, 2022, which claims the benefit of each of U.S. Provisional Application No. 63/256,762, filed Oct. 18, 2021, U.S. Provisional Application No. 63/244,321, filed Sep. 15, 2021, and U.S. Provisional Application No. 63/236,743, filed Aug. 25, 2021; and is a continuation-in-part of U.S. application Ser. No. 18/371,795, filed Sep. 22, 2023, which is a continuation of U.S. application Ser. No. 17/861,437, filed Jul. 11, 2022, now U.S. Pat. No. 11,801,001, which is a continuation of U.S. application Ser. No. 17/306,099, filed May 3, 2021, now U.S. Pat. No. 11,412,967, which claims the benefit of U.S. Provisional Application No. 63/140,534, filed Jan. 22, 2021, and which is a continuation-in-part of U.S. application Ser. No. 16/739,441, filed Jan. 10, 2020, now U.S. Pat. No. 11,383,013, which is a continuation of International Application No. PCT/US2019/033318, filed May 21, 2019, which claims the benefit of each of U.S. Provisional Application No. 62/846,400, filed May 10, 2019, U.S. Provisional Application No. 62/752,480, filed Oct. 30, 2018, and U.S. Provisional Application No. 62/674,144, filed May 21, 2018, all of which applications are incorporated by reference herein in their entireties.

### BACKGROUND

The present application relates to systems and methods for performing plasmapheresis and, more particularly, to plasmapheresis systems and methods in which the volume of plasma that may be collected from a particular donor is optimized.

Plasmapheresis is an apheresis procedure in which whole blood is withdrawn from a donor, plasma is separated from other cellular blood components (red blood cells, platelets, and leukocytes) and retained, and the cellular blood components are returned to the donor. The separation of the plasma from the cellular components may be accomplished in an automated procedure by centrifugation or membrane filtration.

The FDA issued guidelines for registered blood collection centers as to the volume of plasma that may be collected as source plasma during plasmapheresis. (FDA Memo: “Volume Limits-Automated Collection of Source Plasma (Nov. 4, 1992)”). The FDA Memo set forth a simplified plasma volume nomogram, in which the volume (or weight) of pure (or raw) plasma that may be collected from a particular donor is limited.

Because the source plasma from multiple donors can be combined, it is advantageous to maximize the plasma volume that may be collected from each individual donor, as even small gains in volume collected from each individual donor, when added together, result in a meaningful increase in the total volume of the pooled plasma.

### SUMMARY

In one embodiment, a system for collecting plasma comprises a reusable component having a control circuit configured to control operation of the system. The control circuit may be coupled to an input device and configured to receive one or more donor parameters and to estimate a physiological fluid amount of the donor based at least in part on the one or more donor parameters. The control circuit may be configured to calculate a target amount of plasma product comprising raw plasma and anticoagulant by multiplying a prestored constant by the estimated physiological fluid amount. The control circuit may be configured to control the system to operate draw and return phases to withdraw whole blood from a donor and separate the whole blood into plasma product and a second blood component and to return the second blood component to the donor. The control circuit may be configured to operate the draw and return phases until a measured amount of plasma product in the collection container meets the target amount of plasma product.

In one embodiment, a system for collecting plasma comprises a separator configured to separate whole blood into a plasma product and a second blood component comprising red blood cells, the blood separator having a plasma output port coupled to a plasma line configured to send the plasma product to a plasma product collection container. A donor line may be configured to introduce the whole blood from the donor to the separator, flow through the donor line being controlled by a first pump. An anticoagulant line may be coupled to an anticoagulant source, flow through the anticoagulant line being controlled by a second pump to combine anticoagulant with the whole blood from the donor based on an anticoagulant ratio. A touchscreen may be configured to receive input from an operator. A control circuit may be configured to control operation of the system, the control circuit coupled to the touchscreen and configured to receive a weight of a donor. The control circuit may be configured to calculate an extracellular fluid amount of the donor based at least on the weight of the donor and to calculate a target volume for plasma product and/or raw plasma based at least in part on the extracellular fluid amount. The control circuit may be configured to control the system to operate draw and return phases to withdraw whole blood from a donor and separate the whole blood into the plasma product and the second blood component and to return the second blood component to the donor. The control circuit may be configured to operate the draw and return phases until a volume of plasma product in the collection container equals the target volume for plasma product and/or raw plasma.

In another embodiment, a method for collecting plasma comprises (a) determining the weight of a donor; (b) calculating a donor extracellular fluid amount based, at least in part, on the weight of the donor; (c) calculating a target plasma volume to collect based, at least in part, on a calculated percentage of donor extracellular fluid amount; (d) withdrawing whole blood from the donor through a venous-access device and a first line, the first line connected to a blood component separation device; (e) introducing anticoagulant into the withdrawn whole blood through an anticoagulant line; (f) separating the withdrawn whole blood into a plasma component and at least a second blood component; (g) collecting the plasma component from the blood component separation device and into a plasma collection container; (h) continuing steps (d) through (g) until the target plasma volume to collect is reached in the plasma collection container.

In a first aspect of the present disclosure, a system is provided for collecting plasma from a donor in which the system comprises: a blood separator for separating the whole blood into a plasma product and a second blood component comprising red blood cells, a donor line for introducing whole blood from the donor to the blood separator, a first pump for controlling flow through the donor line, an anticoagulant line coupled to an anticoagulant source for combining anticoagulant with the whole blood, and a second pump for controlling flow through the anticoagulant line.

A touchscreen is provided for receiving input from an operator to a controller programmed to control operation of the system. The controller is configured to determine a target volume of plasma product to be collected (TVPP), either based on the weight of the donor and the donor hematocrit, or based on the weight and height of the donor and the donor hematocrit, to control the system to operate a draw and return cycle to withdraw whole blood from the donor, to add anticoagulant to the whole blood at a pre-determined ratio (ACR), to separate the anticoagulated whole blood into the plasma product and the second component and to return the second component to the donor, and to stop withdrawing whole blood from the donor and initiate a final return of the second blood component when a measured volume of plasma product in a plasma collection container reaches the target volume for plasma product.

In a second aspect, the controller is programmed to calculate i) a target volume of pure plasma to be collected (TVP) based on the weight of the donor and ii) a percentage of anticoagulant in the target volume of plasma product to be collected (% AC_{TVPP}) based on the pre-determined anticoagulant ratio, ACR, and the donor hematocrit, wherein the TVPP=TVP/(1−% AC_{TVPP}), with % AC_{TVPP} expressed as a fraction.

In a third aspect, the controller is programmed to calculate a total blood volume of the donor (TBV) based on the weight and height of the donor, a target volume of pure plasma to be collected (TVP) as a percentage of the TBV, and a percentage of anticoagulant in the target volume of plasma product to be collected (% AC_{TVPP}) based on the pre-determined anticoagulant ratio (ACR) and the donor hematocrit, wherein the TVPP=TVP/(1−% AC_{TVPP}/100), with % AC_{TVPP} expressed as a fraction.

In a fourth aspect, the controller is programmed to calculate the total blood volume of the donor (TBV) based on the weight and height of the donor to calculate a body mass index for the donor (BMI) such that TBV=(Weight*70)/(sqrt(BMI/22)) (Lemmens equation). In alternative embodiments, calculations or estimations of total blood volume other than the Lemmens equation may be used.

In a fifth aspect, the controller is programmed to calculate the total blood volume of the donor (TBV) based on the weight (Wt), height (Ht) and sex (Male or Female) of the donor such that TBV=(0.3669*Ht³)+(0.03219*Wt)+0.6041 for Males and TBV=(0.3561*Ht³)+(0.03308*Wt)+0.1833 for Females, where Ht is in meters and Wt is in kilograms (Nadler's formula).

In a sixth aspect, methods are provided for performing plasmapheresis to collect a volume of plasma product (i.e., anticoagulated plasma, VPP) so that that the targeted volume of pure plasma (TVP) in the plasma product is determined based on donor-specific characteristics, consistent with the donor's safety and comfort. In particular, the targeted volume of pure plasma to be collected, TVP, is based on the weight, or the weight and the height, of the donor.

In a seventh aspect, the targeted volume of pure plasma to be collected, TVP, may be a multiple or fraction of the donor's weight. Alternatively, TVP may be a multiple of the donor's total blood volume, TBV, with the TBV of the donor being determined based on the donor's weight and height, using well established methodology, such as the Lemmens equation or Nadler's formula.

A target volume for the plasma product to be collected, TVPP, is established based on the target volume/weight of pure plasma and the percentage of anticoagulant, AC, in the plasma product, % AC_{TVPP}, such that TVPP=TVP/(1−% AC_{TVPP}/100), with % AC_{TVPP} expressed as a fraction, wherein % AC_{TVPP} is based on an AC ratio, ACR, and the hematocrit of the donor.

Once the TVPP is determined, the plasmapheresis procedure is commenced, with whole blood being drawn from the donor, mixed at a specified ratio with anticoagulant, and then separated into plasma, red blood cells, and other cellular components. Once the TVPP has been collected, as determined by, e.g., a weigh scale associated with a plasma collection container, the withdrawal of whole blood from the donor ceases, and the red blood cells and other cellular components are returned to the donor.

In a seventh aspect, in determining the target amount for the plasma product to be collected, the hematocrit of the donor may be determined prior to the collection phase of each cycle, either by calculation or on the basis of a signal from a sensor or the like that is indicative of the donor's hematocrit. Further, the amount of plasma product in the plasma collection container may be determined by, e.g., a weigh scale associated with the plasma collection container or an optical sensor that directly measures the volume.

In other aspects, a method is provided for operating a plasmapheresis system to collect a plasma product volume that comprises the maximum allowable volume/weight of raw plasma in accordance with the limits set forth in the FDA nomogram based on the weight of the donor.

In order to collect the maximum volume/weight of raw plasma permitted by the FDA nomogram, a modified nomogram is provided that utilizes the donor's hematocrit to calculate a target volume/weight for a plasma product having the maximum volume of raw plasma permitted by the FDA nomogram. A calculated volume/weight of raw plasma is compared to the maximum volume/weight for the raw plasma permitted by the FDA nomogram. If the calculated volume/weight of raw plasma is less than the maximum permitted volume/weight, the volume/weight of the plasma product to be collected is adjusted upward from the maximum volume/weight permitted by the FDA nomogram for the plasma product by an amount equal to the difference plus the additional amount of anticoagulant that is added to process the additional volume/weight of plasma.

Thus, with the knowledge of the donor's hematocrit and the instrument's AC ratio, the volume of additional raw plasma that may be safely collected from the donor consistent with the limits set forth in the FDA nomogram is determined, and then the total volume/weight of plasma product to be collected based on the weight of the donor set forth in the FDA nomogram is adjusted accordingly.

Plasmapheresis procedures described herein may involve sequential cycles of alternating phases, one in which whole blood is withdrawn from the donor and the plasma separated and collected, and the other in which the separated red blood cells and any other non-RBC cellular components are returned to the donor. The donor's hematocrit will change during the course of the plasmapheresis procedure, thus affecting the amount of anticoagulant in the plasma product collected from one cycle to the next.

In the first aspect of the disclosure, before the commencement of the subsequent extraction/separation phase, a new hematocrit value for the donor is determined, and the target volume/weight of plasma product for the procedure is recalculated before the commencement of each extraction/separation phase to ensure that the maximum amount of raw plasma permitted by the FDA nomogram is collected.

In another aspect, a further method for collecting a volume of plasma during an apheresis procedure is provided. The steps of the method comprise: determining a total whole blood volume V_{b} for the donor; determining a volume of raw plasma (V_{RP}) that may be collected from the donor based on V_{b}; determining a target volume of plasma product (V_{PP}) to be collected, wherein V_{PP} is equal to the volume of raw plasma (V_{RP}) to be collected plus a volume of anticoagulant (V_{AC}) that is added to the V_{RP} during the apheresis procedure, such that V_{PP}=V_{RP}*K, where K=(ACR*(1−Hct/100)+1)/(ACR*(1−Hct/100)), based on an anticoagulant ratio (ACR, defined as the ratio of donor blood volume to anticoagulant volume for donor blood having no anticoagulant) established for the procedure and a Hct of the donor; withdrawing whole blood from the donor; adding anticoagulant to the whole blood in an amount consistent with the ACR; separating plasma product from the whole blood; and transferring the plasma product to a collection container until the volume of plasma product in the collection container reaches V_{PP}. Because the plasmapheresis procedure comprises multiple extraction/separation and return phases, the V_{PP} for the procedure is recalculated before each extraction/separation phase is commenced, based on a value for the hematocrit of the donor determined prior to the start of each draw phase, and the target volume for the plasma product adjusted accordingly. Alternatively, V_{RP} may be determined based on a calculated value for the donor's total plasma volume, based on V_{b} and the donor's hematocrit.

In another aspect, a method for determining a volume of plasma product (V_{PP}) that may be collected during an apheresis procedure is provided, wherein V_{PP} is equal to a volume of raw plasma (V_{RP}) that may be collected plus a volume of anticoagulant (V_{AC}) that is added to the V_{RP} during the apheresis procedure. The steps of the method comprise: determining a weight (W_{kg}) and sex (M or F) of the donor, determining a hematocrit (Hct) for the donor; determining the volume of raw plasma (V_{RP}) that may be collected based on the weight (W_{kg}) and sex (M or F) of the donor; determining a ratio K between the V_{PP} and the V_{RP}, such that K=V_{PP}/V_{RP}, based on an anticoagulant ratio (ACR) and the Hct of the donor; determining V_{PP}, such that V_{PP}=V_{RP}*K. Further, K=(ACR*(1−Hct/100)+1)/(ACR*(1−Hct/100)). After V_{PP} is determined, whole blood is withdrawn from the donor; anticoagulant is added to the whole blood in an amount consistent with the ACR; plasma product is separated from the whole blood; and plasma product is transferred to a collection container. After a desired amount of whole blood has been withdrawn from the donor, the red blood cells are returned to the donor. Then, the Hct of the donor and V_{PP} are determined prior to each draw phase.

In a related aspect, the draw and separation steps are repeated until the volume of plasma product in the collection container reaches V_{PP}.

In a related aspect, the donor's hematocrit subsequent to the first collection phase may be calculated by a volume balance, assuming that the donor's quantity of red blood cells is the same at the start of each draw cycle, while the total volume of blood decreases from one cycle to the next in an amount equal to the amount of raw plasma collected. Alternatively, the donor's hematocrit at the start of each draw cycle can be measured by an optical or other sensor.

In a further aspect, the volume of raw plasma that may be collected from a particular donor may be determined by any one of several different means. Such means include, e.g., the FDA nomogram, taking into account only the donor's weight; a modified FDA nomogram, further taking into account the donor's hematocrit, and taking a fraction of a total blood volume or total plasma volume calculated for a particular donor. The total blood volume or total plasma volume may be determined using, for example, Nadler's equations, Gilcher's Rule of Five, tables provided by the International Council for Standardization in Haematology (ICSH), or any other generally accepted method using the donor's height, weight, sex, and age, consistent with the safety and comfort of the donor.

In another aspect, an automated system for separating plasma from whole blood is provided that comprises a reusable hardware component and a disposable kit. The disposable kit further comprises i) a separator for separating whole blood into a plasma fraction and a concentrated cell fraction, the separator having an input having a blood line integrally connected thereto for transporting whole blood from a donor to the separator, a plasma output port integrally connected to a plasma collection container by a plasma line, and a concentrated cell outlet port integrally connected to a reservoir for receipt of concentrated cells prior to reinfusion to the donor; ii) a donor line terminating in a venipuncture needle for transporting whole blood from a donor to the blood line, iii) an anticoagulant line integrally connected to the blood line and configured to be connected to a source of anticoagulant for transporting anticoagulant to the donor line, and iv) a reinfusion line for transporting concentrated cells from the reservoir to the donor line.

The reusable hardware component further comprises i) a first peristaltic pump for delivering anticoagulant at a controlled rate into the blood line during a collection phase, ii) a second pump for delivering anticoagulated whole blood to the separator during the collection phase and for returning concentrated cellular components during a reinfusion phase, iii) a third pump for delivering concentrated cellular components from the separator to the reservoir during the collection phase, iv) a clamp associated with each of the blood line, plasma line, and reinfusion line, v) a weigh scale for weighing each of the plasma collection container, the reservoir and the source of anticoagulant, and vi) a programmable controller comprising a touch screen for receiving input from an operator, the programmable controller configured to receive a signal from each of the weigh scales and to automatically operate the first, second and third pumps and the clamps to separate whole blood into a plasma fraction and a concentrated cell fraction during the collection phase and to return concentrated cells to the donor during the reinfusion stage. The programmable controller is further configured to determine a target amount for the plasma product to be collected in the plasma collection container in accordance with any of the methods described herein, and to terminate the collection phase upon receiving a signal that the amount of plasma product in the plasma collection container equal to the target amount of the plasma product determined by the controller. In determining the target amount for the plasma product to be collected, the controller may be configured to calculate the hematocrit of the donor prior to the collection phase of each cycle. Alternatively, or additionally, the controller may receive a signal from a sensor or the like that is indicative of the donor's hematocrit. Further, the amount of plasma product in the plasma collection container may be determined by, e.g., the weigh scale associated with the plasma collection container or an optical sensor that directly measures the volume.

### BRIEF DESCRIPTION OF THE DRAWINGS

FIG. 1 is a perspective view of an exemplary plasmapheresis instrument suitable for use in the system and method of the present application.

FIG. 2 is a perspective view of a spinning membrane separator of the type incorporated in a disposable set, with portions broken away to show detail, usable with the plasmapheresis system of FIG. 1.

FIG. 3 is a perspective view of the front panel of the plasmapheresis system of FIG. 1 showing the components of the disposable set that are mounted thereto.

FIG. 4 is a schematic view showing operation of the plasmapheresis system in the collection phase.

FIG. 5 is a schematic view showing operation of the plasmapheresis system in the reinfusion phase.

FIGS. 6a and 6b are flow charts showing the steps of methods used in the present application for collecting a target volume of pure plasma.

FIG. 7 is a table that shows the volume of pure plasma, based on donor hematocrit, that is contained within a plasma product volume limit set by the FDA nomogram using a 1:16 ratio of anticoagulant to whole blood.

FIG. 8 is a table that shows the volume of “unclaimed” pure plasma in the plasma product based on the difference between the values set forth in FIG. 7 and the maximum volume of pure plasma that may be collected based on the FDA nomogram.

FIG. 9 is a table that shows the volume of plasma product that may be collected from a donor, based on the donor's weight and hematocrit, that results in the maximum permissible volume of pure plasma permitted by the FDA nomogram.

FIG. 10 is a table showing the inputs to a programmable controller for performing a hypothetical plasmapheresis procedure in accordance with the method of the present application.

FIGS. 11a, 11b comprise a table, broken into two parts illustrating how the donor's hematocrit increases over the course of a hypothetical plasmapheresis procedure based on the inputs from the table of FIG. 10, and resulting in an increase in the total collection volume of plasma product necessary to collect the target volume of pure plasma.

FIG. 12 is a graph illustrating IgG dilution during plasmapheresis.

FIG. 13 is a flowchart illustrating a method of collecting plasma using extracellular fluid amount, according to another exemplary embodiment.

FIG. 14 is a flowchart illustrating a method of collecting plasma using an estimate of a physiological fluid amount and a prestored constant, according to another exemplary embodiment.

FIGS. 15A and 15B are charts plotting percentage of ECFV removed as plasma against incremental plasma volume achieved over a prior nomogram.

FIG. 16 is a table showing change in plasma collected using fixed Hct values versus actual donor Hct.

FIG. 17 is a table showing prestored constants for different nomograms using fixed Hct values.

FIG. 18 is a table showing exemplary nomograms for various different embodiments.

FIG. 19 shows exemplary blood volume formulas for various different embodiments.

### DETAILED DESCRIPTION

A more detailed description of the systems and methods in accordance with the present disclosure is set forth below. It should be understood that the description below of specific devices and methods is intended to be exemplary, and not exhaustive of all possible variations or applications. Thus, the scope of the disclosure is not intended to be limiting and should be understood to encompass variations or embodiments that would occur to persons of ordinary skill. Various aspects of the system and method are described in greater detail in US 2020/0147289, which is incorporated herein by reference.

In some embodiments, setting a target plasma volume to collect to be a percentage of donor extracellular fluid volume may improve the consistency of risk across different donors.

In some embodiments, setting a target plasma volume to collect to be a percentage of donor extracellular fluid volume may improve the consistency of the collected plasma product.

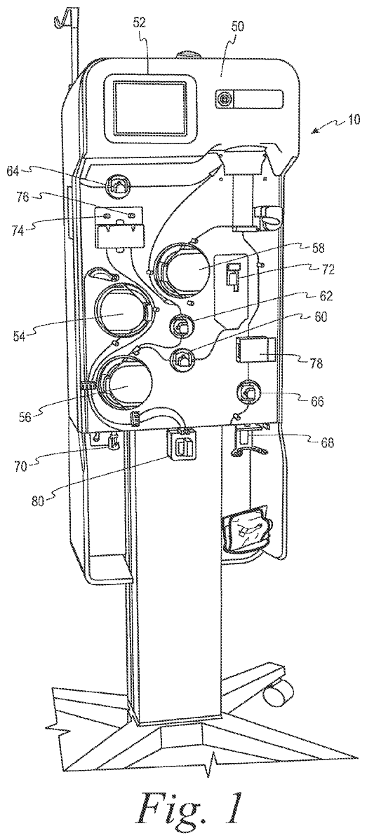

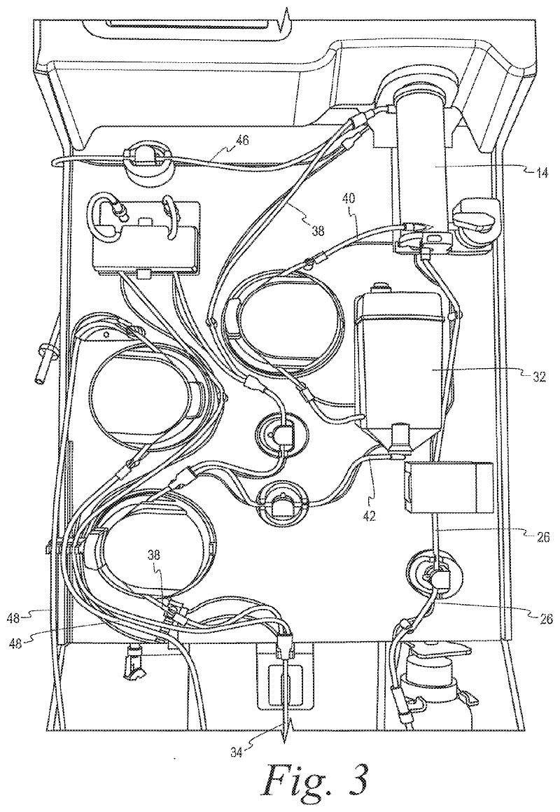

In the context of the present application, plasmapheresis is performed on an automated system comprising a hardware component, generally designated 10, and a disposable set, generally designated 12, to collect plasma to be processed as source plasma. With reference to FIGS. 1-5, and as described in greater detail below, the disposable set 12 consists of an integrally connected separator, containers, and tubing to transport blood and solutions within a sterile fluid pathway.

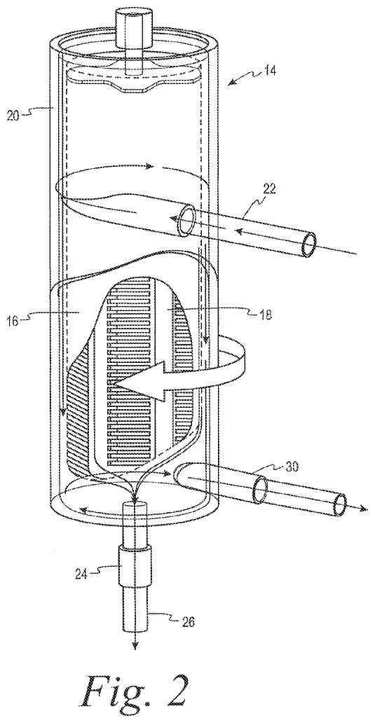

The separator 14, best seen in FIG. 2, has a spinning membrane filter 16 mounted to a rotor 18 for rotation within a case 20 to separate blood into components. A detailed description of a spinning membrane separator may be found in U.S. Pat. No. 5,194,145 to Schoendorfer, which is incorporated herein by reference. As can be appreciated, in a different system, separation of the whole blood may be accomplished by centrifugation. See, e.g., U.S. Pat. No. 5,360,542 to Williamson et al.

During plasmapheresis, anticoagulated whole blood enters the separator 14 through a whole blood input port 22. The plasma is separated by the spinning membrane filter and then passes out of a plasma output port 24, through a plasma line 26, and into a plasma collection container 28. Concentrated cells are pumped out of a concentrated cell output port 30 into a reservoir 32, where the cells remain until reinfusion to the donor.

The disposable set 12 also includes tubing lines for introducing whole blood from the donor into the system during collection and returning concentrated cells to the donor during reinfusion (donor line 34, which terminates in the venipuncture needle 36), and for transporting anticoagulated whole blood to the separator (blood line 38), concentrated cells into the reservoir (cell line 40), concentrated cells from the reservoir to the donor line (reinfusion line 42), plasma into the plasma collection container (plasma line 44), saline (saline line 46), and anticoagulant (AC line 48).

The hardware component 10 includes a programmable controller 50 and touch screen 52 with a graphical user interface (“GUI”) through which the operator controls the procedure. For example, the GUI permits entry of any of a donor ID, donor sex, donor height, donor weight, donor age, donor hematocrit/hemoglobin; a target saline infusion volume (if a saline protocol is selected), and a target plasma volume. The touch screen 52 also enables the operator to gather status information and handle error conditions.

Three peristaltic pumps are located on the front panel of the hardware component 10, including an AC pump 54, a blood pump 56, and a cell pump 58. The AC pump 54 delivers anticoagulant solution (AC) at a controlled rate into the blood line 38 as whole blood enters the set from the donor. The blood pump 56 delivers anticoagulated whole blood to the separator during the collection phase of the procedure and returns concentrated cellular components and, if desired, replacement fluid to the donor during the reinfusion phase of the procedure. The cell pump 58 delivers concentrated cellular components from the separator 14 to a reservoir during the collection phase.

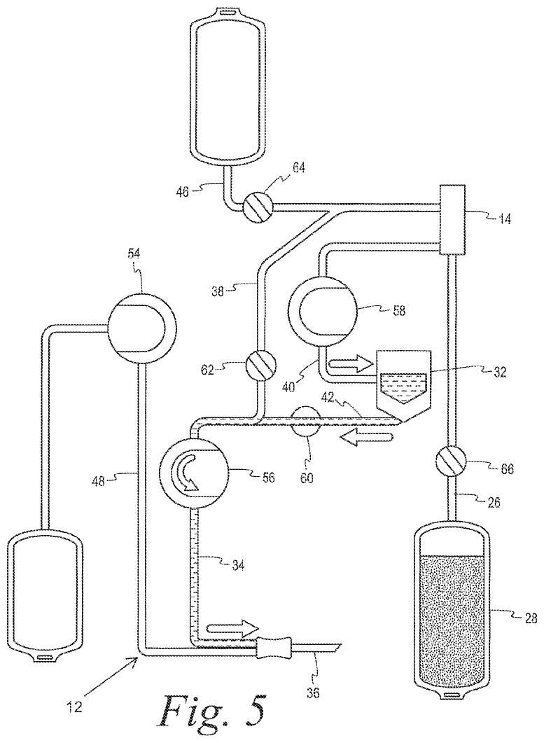

The front panel also includes four clamps into which tubings from the disposable set 12 are installed, including a reinfusion clamp 60, a blood clamp 62, a saline clamp 64, and a plasma clamp 66. The reinfusion clamp 60 closes to block the reinfusion line (42) during the collection phase (FIG. 5) and is open during the reinfusion phase (FIG. 5) to allow the blood pump to reinfuse the concentrated cellular components from the reservoir 32 to the donor. The blood clamp 62 opens during the collection phase to allow anticoagulated whole blood to be pumped to the separator 14 and closes during the reinfusion phase to block the blood line 38. The saline clamp 64 closes to block the saline line 46 during the collection phase and during reinfusion of the separated cellular components. If saline is to be used as a replacement fluid, the saline clamp 64 opens during the reinfusion phase. The plasma clamp 66 opens during the collection phase to allow plasma to flow into the plasma collection container 28 and closes during the reinfusion phase.

The hardware component 10 includes three weigh scales to monitor the current plasma collection volume (scale 68), the AC solution volume (scale 70), and the concentrated cellular content volume (scale 72). The system also includes various sensors and detectors, including a venous pressure sensor 74, a separator pressure sensor 76, optical blood detectors 78, and an air detector 80.

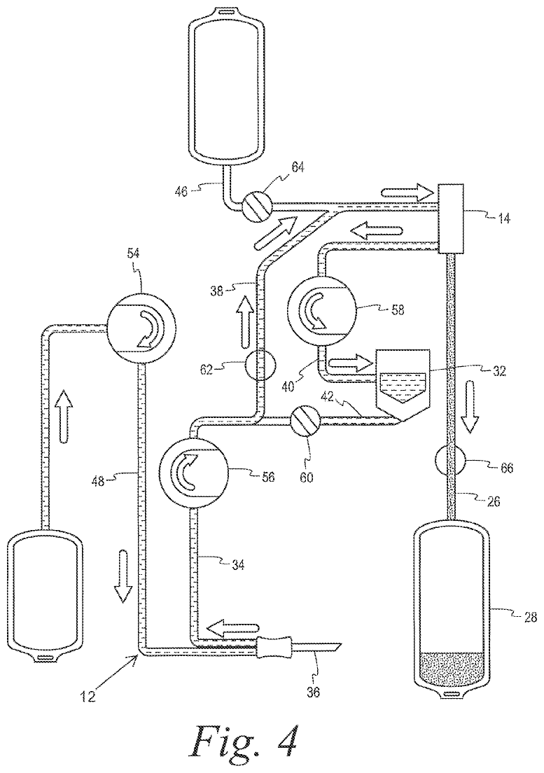

The donor is connected to the system throughout the procedure. As illustrated, the disposable set 12 includes a single venipuncture needle 36, through which whole blood is drawn from the donor in a collection phase (FIG. 4) and concentrated cells are returned to the donor in a reinfusion stage (FIG. 5). As noted above, the plasmapheresis procedure may comprise a plurality of cycles each having a collection/separation phase followed by a return or reinfusion phase. During the collection phase, the whole blood is separated into plasma and concentrated cells. The disposable set includes a plasma collection container 28 for receipt of the separated plasma and a reservoir 32 for receipt of the concentrated cells. During the reinfusion phase, the concentrated cells from the reservoir 32 are reinfused to the donor through the venipuncture needle 36. Plasmapheresis performed with a single venipuncture needle 36 may involve multiple cycles of collection and reinfusion.

Returning to FIG. 4, during the collection phase, anticoagulant solution (AC) is pumped at a controlled rate and mixed with whole blood as it enters the disposable set 12. The anticoagulated blood is pumped to the separator 14, where plasma is separated from the cellular components and directed to the plasma collection container 28.

The cellular components are pumped from the separator 14 to the reservoir 32. The collection phase stops when the reservoir 32 reaches an expected volume of concentrated cells or if the target plasma collection volume has been achieved.

Then, the reinfusion phase begins. With reference to FIG. 5, during the reinfusion phase, the blood pump 56 reverses direction and pumps the concentrated cells from the reservoir 32 back to the donor through the apheresis needle 36. If a saline protocol was selected, by which saline is returned to the donor as a replacement fluid for the collected plasma, the final reinfusion phase is followed by saline infusion.

The automated plasma collection device is configured to collect a volume/weight of anticoagulated plasma (i.e., the plasma product) having the maximum volume/weight of raw plasma permitted for the donor under the limits set forth in the FDA nomogram. In order to maximize the volume of raw plasma comprising the plasma product, the device is programmed with a nomogram that accounts for the donor's hematocrit. With the knowledge of the donor's hematocrit and the instrument's AC ratio, the total volume/weight of plasma product to be collected can be determined such that the plasma product includes the maximum volume/weight of raw plasma fraction that may be collected from a donor, consistent with the limits for total volume/weight of raw plasma set forth in the FDA nomogram. By having the computations programmed into the controller, the likelihood of operator error is diminished in comparison to the off-line calculation of the collection volume that is then entered into the instrument.

During plasmapheresis, when anticoagulant is mixed with whole blood as it is drawn from the donor, the anticoagulant is evenly distributed within the pure/raw plasma in the blood. However, the amount of pure/raw plasma in the whole blood is dependent on the hematocrit (Hct) of the whole blood. The following relationships are established:

$$Volume of R B C = Volume of Whole Blood * Hct / 100. \qquad [ 1 ]$$

$$Volume of Pure / Raw Plasma = Volume of Whole Blood * ( 1 - Hct / 100 ) . \qquad [ 2 ]$$

When anticoagulant is mixed with the whole blood, it may be metered at an AC Ratio (ACR) of 16 parts of whole blood to 1 part of AC, or at 1 part of whole blood to 0.0625 parts of AC.

$$A C R = Volume of Whole Blood / Volume of Anticoagulant ( the donor blood having no anticoagulant ) . \qquad [ 3 ]$$

(This yields a slightly different result from the FDA nomogram, which, as noted above, standardizes the volume of anticoagulant that may be added to a 1:16 ratio of anticoagulant to anticoagulated blood, or 0.06 parts anticoagulant to 1 part anticoagulated blood.)

$$Volume of Anticoagulated Blood = Volume of Anticoagulant + Volume of Whole Blood . \qquad [ 4 ]$$

Combining equations gives:

$$Volume of Pure / Raw Plasma = A C R * Volume of Anticoagulant * ( 1 - Hct / 100 ) . \qquad [ 5 ]$$

Since the red cells are given back to the donor:

$$Volume collected Plasma Product = Volume of Pure / Raw Plasma + Volume of Anticoagulant . \qquad [ 6 ]$$

Equations [5] and [6] can be combined to calculate the amount of anticoagulant in a given amount of collected plasma:

$$Volume of Anticoagulant = Volume of collected Plasma Product / ( 1 + ACR * ( 1 - Hct / 100 ) ) . \qquad [ 7 ]$$

### Further:

$$Volume of collected Plasma Product = Volume of Pure / Raw Plasma * K , where K = ( ACR * ( 1 - Hct / 100 ) + 1 ) / ( ACR * ( 1 - Hct / 100 ) ) . \qquad [ 8 ]$$

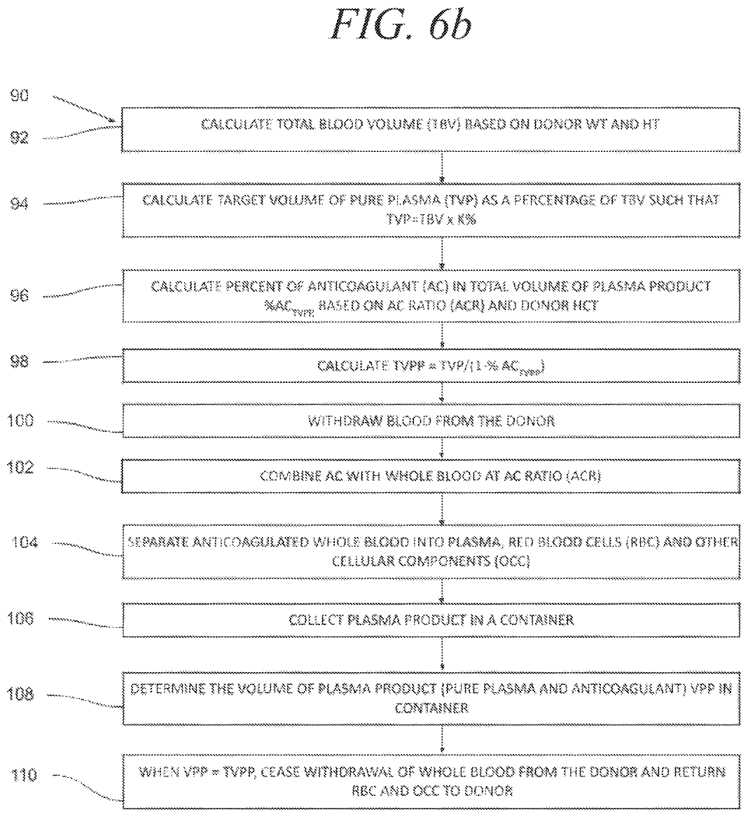

In view of the relationships expressed in the equations above, the volume of pure/raw plasma contained within the volume of plasma product permitted under the FDA nomogram can be determined based upon the hematocrit of the donor. The results of such calculations are set forth in FIG. 7, which shows the volume of pure/raw plasma based on donor hematocrit that is contained within a plasma product volume limit set by the FDA nomogram.

As can be appreciated with reference to FIG. 7, for donors weighing from 110 to 149 lbs. (for whom the maximum plasma product volume per the FDA nomogram is 690 mL), if the donor has a hematocrit of 42 or greater, the volume of raw plasma collected is less than the 625 mL permitted by the FDA nomogram. The situation is similar for donors having a weight of 150 to 174 lbs. (for whom the maximum plasma collection volume per the FDA nomogram is 825 mL) and for donors having a weight of 175 lbs. and up (for whom the maximum plasma collection volume per the FDA nomogram is 880 mL) when the donor's hematocrit is 40 or greater.

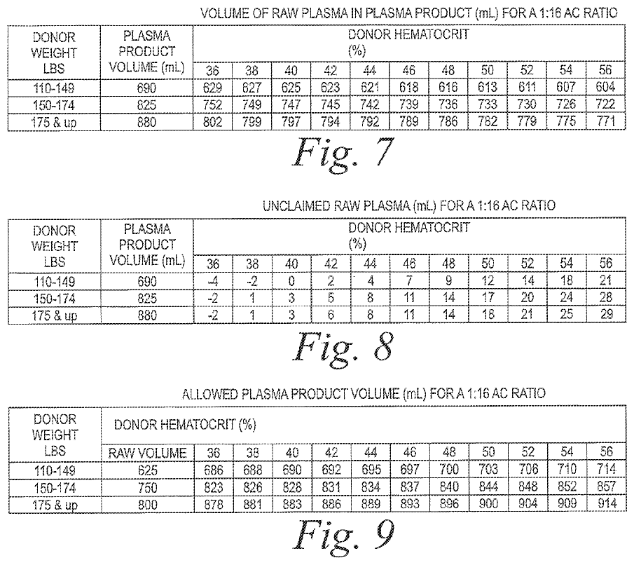

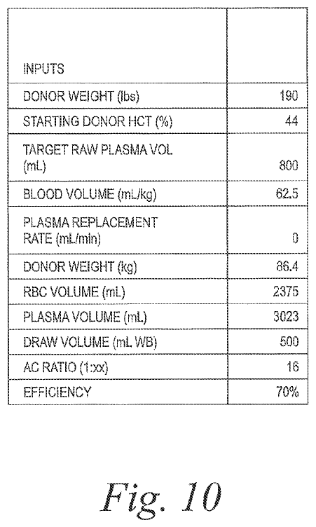

The table set forth in FIG. 8 presents the volume of “unclaimed” raw plasma in the plasma product based the difference between the values set forth in FIG. 7 and the maximum volume of pure/raw plasma that may be collected based on the FDA nomogram. Thus, as shown in the table set forth in FIG. 9, the plasma product collected from any particular donor may be adjusted from that set forth in the FDA nomogram by an amount corresponding to the amount of “unclaimed” pure/raw plasma set forth in FIG. 8 plus the amount of anticoagulant needed to process the additional volume.

Alternatively, the volume of plasma product to be collected may be calculated by first determining a weight and hematocrit (Hct) for the donor; determining the volume of raw plasma (V_{RP}) that may be collected based on the weight of the donor (W_{kg}); determining a ratio K between the V_{PP} and the V_{RP}, such that K=V_{PP}/V_{RP}, based on an anticoagulant ratio (ACR; 1:16 or 0.0625:1, per the FDA nomogram) and the Hct of the donor; and determining V_{PP}, such that V_{PP}=V_{RP}*K. Further, K=(ACR*(1−Hct/100)+1)/(ACR*(1−Hct/100)).

In a further alternative, the volume of plasma product that is to be collected (V_{PP}) may be calculated by first determining the weight (W_{kg}) and hematocrit (Hct) of the donor; determining the volume of raw plasma (V_{RP}) that may be collected based on the weight of the donor (W_{kg}); determining the volume of anticoagulant to be added (V_{AC}) based on the anticoagulant ratio (ACR; 1:16 or 0.06:1, per the FDA nomogram) and the hematocrit of the donor such that V_{AC}=V_{RP}*(ACR*(1−Hct/100)); and determining the collection volume such that V_{PP}=V_{RP}+V_{AC}.

In keeping with one aspect of the disclosure, the automated plasma collection device is configured to collect a volume/weight of plasma product (pure plasma+anticoagulant) having a volume/weight of pure plasma permitted for the donor as determined by either of the two methods set forth in greater detail below.

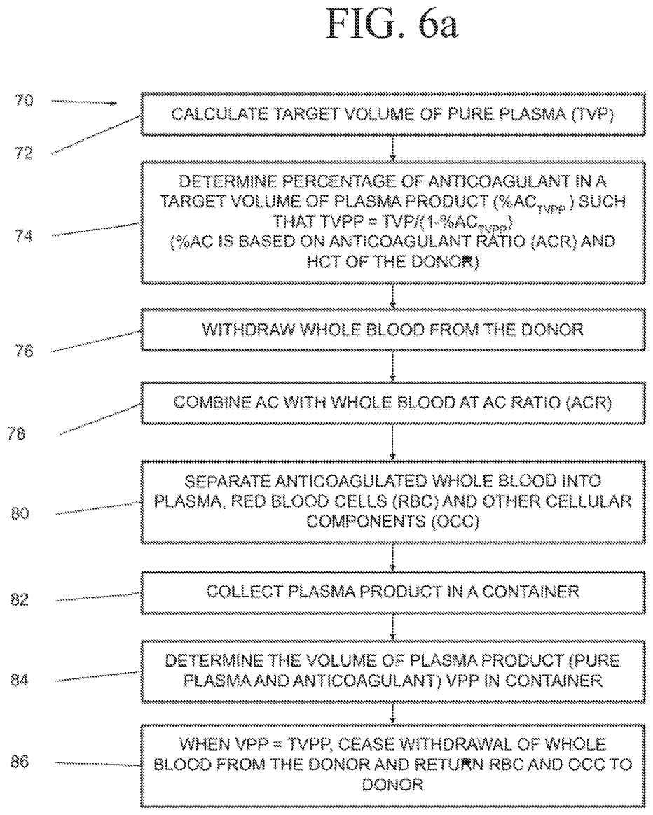

With reference to FIG. 6a, a first method (70) for collecting a target volume of plasma product, TVPP, is illustrated. In the method, the target volume of plasma product (TVPP) is determined by first calculating a target volume of pure plasma to be collected (TVP) based on the weight of the donor (Step 72) and then determining a percentage of anticoagulant in the target volume of plasma product to be collected (% AC_{TVPP}) based on the pre-determined anticoagulant ratio (ACR) and the hematocrit of the donor, wherein the TVPP=TVP/(1−% AC_{TVPP}/100), with % AC_{TVPP} expressed as a fraction (Step 74). In this method no intervening calculation is required of either a total blood volume or a total plasma volume of the donor prior to determining the target collection volume of plasma for the donor, though, in alternate embodiments, such calculations may be included.

Various methods may be used for determining a target volume of pure plasma that may be collected directly from the weight of the donor. For example, the weight of the donor may be multiplied by an established constant “K₁” (such as 10 mL/kg). Alternatively, the weight of the donor may be segregated into weight categories or ranges (e.g., at least three categories, at least six categories, etc.), with a fixed volume established for each category (as in the FDA nomogram discussed above, in which the ranges of donor weight are divided into three categories).

The anticoagulant ratio, ACR, may be defined in one of two different ways. In a first way, ACR₁ is the ratio of the amount of whole blood to the amount of anticoagulant (ACR₁=WB/AC). In a second way, ACR₂ is the ratio of volume of whole blood plus the volume of anticoagulant to the volume of anticoagulant (ACR₂=(WB+AC)/AC). If ACR=WB/AC, then the percent of anticoagulant in the target volume of plasma product, % AC_{TVPP}, is determined according to the following equation: % AC_{TVPP}=1/(1+ACR(1−Hct/100)), with Hct being expressed as a percentage. If ACR=(WB+AC)/AC), then the percent of anticoagulant in the target volume of plasma product, % AC_{TVPP}, is determined according to the following equation: % AC_{TVPP}=1/(1+(ACR-1)(1−Hct)). The ACR may be expressed as either a ratio or a percentage and may vary from 7:1 to 20:1, or from about 5% to 14%. An exemplary ACR is 16:1, or 6.25%.

Returning to FIG. 6a, once the TVPP has been determined, as described above, whole blood is withdrawn from the donor (Step 76) and combined with anticoagulant based on a predetermined ratio, ACR (Step 78). Anticoagulated whole blood is then introduced into separator 14, where it is separated into plasma and concentrated (red blood) cells (Step 80). Plasma product (pure plasma and anticoagulant) is collected in plasma collection container 28 (Step 82) while separated red blood cells are collected in reservoir 32. As the plasma product is being collected, the volume of plasma product, V_{PP}, (pure plasma and anticoagulant) in the plasma container is determined (Step 84). When the V_{PP} equals the Target volume of plasma product (TVPP), withdrawal of whole blood ceases and any remaining blood components (such as red blood cells) are returned to the donor (Step 86).

With reference to FIG. 6b, a second method (90) for collecting a target volume of plasma product, TVPP, is illustrated. In this method, the target volume of plasma product, TPPV, is determined by first calculating a total blood volume of the donor (TBV) based on the weight and height of the donor (Step 92), calculating a target volume of pure plasma to be collected, TVP, as a percentage of the TBV (Step 94), and calculating a percentage of anticoagulant in the target volume of plasma product to be collected (% AC_{TVPP}) based on the pre-determined anticoagulant ratio (ACR) and the hematocrit of the donor (Step 96), and calculating TVPP wherein TVPP=TVP/(1−% AC_{TVPP}/100), with % AC_{TVPP} expressed as a fraction (Step 98). The % AC_{TVPP} may be determined as described above in connection with the first method. In this method no calculation of a total plasma volume of the donor is required to determine the target collection volume of plasma for the donor.

A donor's plasma volume may be estimated based on the donor's total blood volume, and a volume of plasma that may be harvested consistent with donor safety and comfort may be based on this estimation. Methods utilizing donor parameters may be used to estimate a donor's total blood volume. The donor's total blood volume may be determined using one or more of Lemmens equation (that uses the donor's body mass index to determine a total blood volume), Nadler's equations (that take into account the height, sex and weight of the donor), Gilcher's Rule of Five (that takes into account sex, weight and morphology (obese, thin, normal or muscular), or the standards of the International Counsel for Standardization in Haematology (“ICSH) as set forth in Br. J. Haem. 1995, 89:748-56) (that consider the height, weight, age, and sex of the donor). Any other methodology for determining donor's total blood volume may also be used, such as that disclosed in Lemmens et al, “Estimating Blood Volume in Obese and Morbidly Obese Patients,” Obesity Surgery, 16, 773-776, 2006:

$$InBv = \frac{70}{\sqrt{BMIp / 22}}$$

in which _{In}Bv is indexed blood volume, that is, the blood volume per unit mass of the donor, and BMIₚ is the body mass index of the donor, based on donor weight and height. An age-dependent regression equation may also be used for indexed blood volume _{In}BV at IBW (ideal body weight), as shown below:

$${}_{In} BV = 90 - 0.4 \times age ( males ) ; {}_{In} BV = 85 - 0.4 \times age ( females )$$

Thus, the indexed blood volume may be calculated based on sex of the donor.

In another embodiment, a plurality of such methodologies may be used and the average, mean, or a weighted average of the methodologies may be taken as the donor's total blood volume. For example, once the donor's total blood volume is determined, the donor's plasma volume may be estimated by multiplying the total blood volume by a constant “K₂”, where or K₂ equals (1−Hct of the donor).

From an analysis of demographic, examination, and laboratory data from the 2015-2016 National Health and Nutrition Examination Survey, in which sex, age, height, weight, pregnancy data and hematocrit were extracted, along with the formulas in Pearson et al., Interpretation of measured red cell mass and plasma volume in adults: Expert Panel on Radionuclides of the International Council for Standardization in Haematology, British J. Haematology, 89: 748-756 (1995), (upon which the ICSH recommended formulae were derived), it has been determined that for donors having certain characteristics (namely low weight females with high hematocrits), up to 36% of the available plasma may be collected while staying within current regulations. Plasmapheresis procedures with such donors have been carried out routinely without adverse reactions, and thus are considered safe. This suggests that up to 36% of a donor's available plasma can be safely collected in a plasmapheresis procedure.

Given that only negative deviations of a donor's true blood volume from a predicted/calculated total blood volume present a potential risk, a further adjustment downward of the harvestable volume of plasma may be appropriate. Based on a consideration of the deviation between the calculated blood volume as determined in Pearson et al., cited above.

Thus, the total blood volume of the donor (TBV) may be calculated based on the weight (Wt) and height (Ht) of the donor to calculate a body mass index for the donor (BMI) such that TBV=(Weight*70)/sqrt(BMI/22), where BMI=Wt/Ht², and where Ht is in meters and Wt is in kilograms (Lemmens equation). See, “Estimating Blood Volume in Obese and Morbidly Obese Patients,” Lemmens et al., Obesity Surgery 16, 2006, pp. 773-776.

Alternatively, the total blood volume of the donor (TBV) may be calculated based on the weight (Wt), height (Ht) and sex (Male or Female) of the donor such that TBV=(0.3669*Ht³)+(0.03219*Wt)+0.6041 for Males and TBV=(0.3561*Ht³)+(0.03308*Wt)+0.1833 for Females, where Ht is in meters and Wt is in kilograms (Nadler's formula).

The percentage by which TBV is multiplied to obtain TVP (and, ultimately TVPP) is selected to maximize the volume of pure plasma that is collected from the donor consistent with donor comfort and safety. The percentage ranges in various embodiments may be between approximately 1% and 15% of TBV, at least 15%, less than 18%, between about 15% and 17%, about 12%, about 16% or about 18%. The TVPP may also be subject to a maximum volume of, e.g., 1000 mL or 1050 mL to be collected regardless of the donor's TBV.

An adjustment, V_{C}, may be made to the calculated volume of whole blood TBV before calculating the target volume of pure plasma TVP, such that TVP=0.36(1−Hct)(TBV−V_{C}), where V_{C}=523 mL based on a regression analysis of the experimental blood volume data presented in Retzlaff et al., Erythrocyte Volume, Plasma Volume, and Lean Body Mass in Adult Men and Women, J. Haematology, 33, 5:649-667 (1969). There is a 95% confidence that an individual's predicted blood volume will differ not more that 20.5%. Thus, a scaling factor of 0.795 may be applied to determination of harvestable raw plasma being 36% of the donor's total plasma volume described above, so that 28.6% of a donor's calculated volume of raw plasma may be harvested, consistent with donor safety and comfort.

Retuning to FIG. 6b, once the TVPP has been determined, as described above (based on the TBV), whole blood is withdrawn from the donor (Step 100) and combined with anticoagulant based on a predetermined ratio (Step 102). Anticoagulated whole blood is then introduced into separator 14 where it is separated into plasma and concentrated (red blood) cells (Step 104). Plasma product (pure plasma and anticoagulant) is collected in plasma collection container 28 (Step 106) while separated red blood cells are collected in reservoir 32. As the plasma product is being collected, the volume of plasma product, V_{PP}, (pure plasma and anticoagulant) in the plasma container is determined (Step 108). When the V_{PP} equals the Target volume of plasma product (TVPP), withdrawal of whole blood ceases and any remaining blood components (such as red blood cells) are returned to the donor (Step 110).

Thus, the collection volume (the volume of plasma product) is determined based on the volume of raw plasma volume that may be collected from a particular donor, the donor's hematocrit, and the fixed anticoagulant ratio (ACR). Consequently, this methodology allows for more consistent control for the raw plasma volume of the donor, which is the variable most related to donor safety.

In an exemplary method, the operator enters into the system controller the collection volume for the plasma product for the particular donor, based on the target volume of raw plasma that may be harvested. The target plasma collection volume may be as set forth in FIG. 9, based on the donor's weight and hematocrit for the initial collection phase, or by any of the other methods as set forth above. Alternatively, the controller is configured to calculate the target plasma product collection volume for the initial collection phase in accordance with a methodology such as those described above upon the operator entering, e.g., the donor's weight and hematocrit, and/or any of the additional donor-specific information (such as the donor's sex, height and age) required by the methodologies used for determining a donor's total blood volume, total plasma volume, and the target volume of harvestable plasma that may be collected.

In practice, the operator enters into the system controller the collection volume for the plasma product for the particular donor, based on the target volume of raw plasma that may be harvested. The target plasma collection volume may be as set forth in FIG. 9, based on the donor's weight and hematocrit for the initial collection phase, or by any of the other methods as set forth above. Alternatively, the controller is configured to calculate the target plasma product collection volume for the initial collection phase in accordance with a methodology such as those described above upon the operator entering, e.g., the donor's weight and hematocrit, and/or any of the additional donor-specific information (such as the donor's sex, height and age) required by the methodologies used for determining a donor's total blood volume, total plasma volume, and the target volume of harvestable plasma that may be collected.

Preferably, the system administrator will initially set an indication of whether the targeted collection volume of plasma product, TVPP, will be determined by the system (e.g., in accordance with one of the methods described above) or entered directly by the operator into the system. If the operator is to enter the TVPP, then the system administrator will disable the controller's capability to calculate a TVPP. The system administrator will also set an AC ratio to be used for all procedures. If the controller is to determine the TVPP, the administrator will set the system to allow the appropriate donor specific characteristics for calculating the TVPP in accordance with any of the methods described above to be entered into the controller, either by the operator or a donor management system, by which donor parameters used for qualification screening (such as weight, height, and hematocrit) can be electronically sent to the instrument, avoiding operator error in entering the donor parameters. The donor management system could also utilize the donor screening measurements, along with the relationship between pure plasma volume and collection volume, to automatically calculate a TVPP that it would transmit to the controller of the plasmapheresis device. Otherwise, the controller will calculate the TVPP before collection of whole blood form the donor starts. In addition, if the controller/donor management system is to calculate TVPP, the administrator will set the system to enable the operator to enter a TVPP other than the calculated volume. Further, the system will permit the operator to change the TVPP from the calculated TVPP, either before or during the procedure, if, for example, the estimated time for running/completing the procedure needs to be shortened for reasons of donor comfort or convenience. At the completion of the procedure the actual volume of plasma product collected, V_{PP}, and the target volume, TVPP, will be displayed, as well as the actual volume of pure plasma collected and the target volume of plasma, TPV.

As noted above, plasmapheresis procedures may be performed with multiple cycles of collection/draw phases and return/reinfusion phases. If the return/reinfusion phase does not include reinfusion of a replacement fluid, the donor's hematocrit will increase from one cycle to the next. Consequently, if the target volume for plasma product is determined based only on the donor's initial hematocrit, and does not consider the donor's increasing hematocrit, the percentage of anticoagulant in the plasma product will be greater (and the volume of pure plasma less) than what was predicted by the initial calculation for determining the target volume of plasma product. Thus, in order to ensure that the volume of plasma product that is collected contains the maximum volume of raw plasma that was determined to be harvested from a particular donor, the target volume for plasma product is recalculated periodically throughout the plasmapheresis procedure, such as before the start of the collection phase of each cycle, to consider the change in the donor's hematocrit.

Accordingly, after the determination of the target volume for plasma product based on the donor's starting hematocrit is made, the plasmapheresis procedure commences with a first draw phase until a specified volume of whole blood (e.g., approximately 500 mL) has been withdrawn from the donor. Anticoagulant is added to the whole blood and the anticoagulated whole blood is separated into a plasma product, red blood cells, and other non-RBC blood components. At the conclusion of the first draw phase, the red blood cells and non-RBC blood components are returned to the donor. The current volume of plasma product collected after the first draw phase is determined by, e.g., the weigh scale. Then a current value for the hematocrit of the donor is established and a new target volume of plasma product to be collected is determined, and the second cycle of draw and return phases is performed. The cycle of draw and return phases is repeated until the target volume of plasma product for the plasmapheresis procedure is collected, as recalculated prior to the start of each draw phase. After the final collection phase, the controller initiates the final red blood cell reinfusion stage, after which the donor is disconnected.

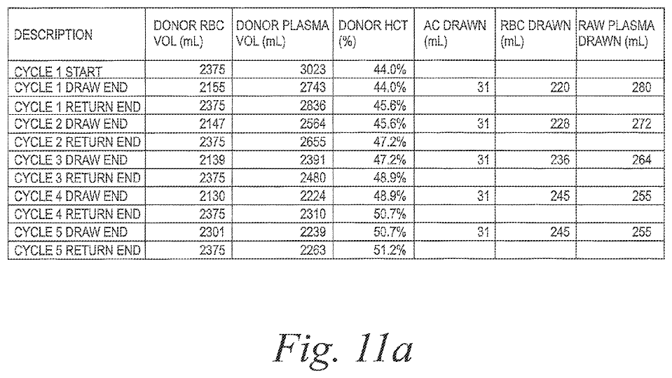

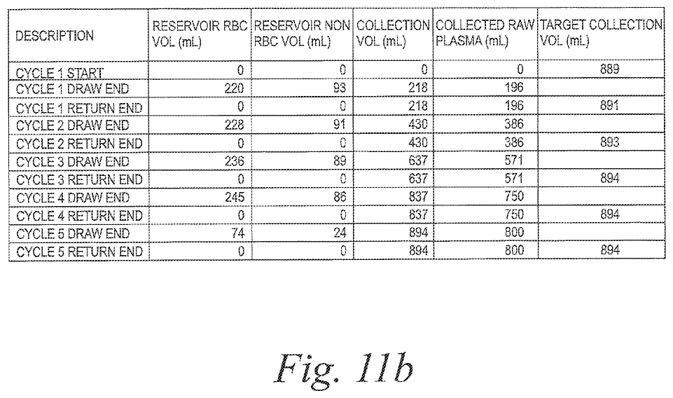

The benefits of performing a plasmapheresis procedure having multiple collection/reinfusion cycles in accordance with the methodology set forth above may be seen by reference to the tables of FIGS. 10 and 11a, 11b. FIG. 10 displays the input data for a hypothetical plasmapheresis procedure for a donor weighing 190 lbs. (86.4 kg) and having an initial hematocrit of 44. With reference to the table of FIG. 1, the simplified FDA nomogram would limit the volume of plasma to be collected from such a donor to 800 mL, and the total collection volume for the plasma product to 880 mL. In the present example, the FDA nomogram limit on the volume of raw plasma that may be collected is for illustrative purposes only. As set forth above, other methodologies may be used to determine the amount of raw plasma that may be safely extracted from a donor that would differ from that indicated by the FDA nomogram.

The number of collection and reinfusion cycles in a plasmapheresis procedure may vary from three to twelve. In the hypothetical plasmapheresis procedure, there are five collection and reinfusion cycles, which are chosen for illustrative purposes.

Before the commencement of the first collection cycle, the volume of raw plasma to be collected and the total target volume of plasma product to be collected are determined in accordance with the methodologies described above, based on the donor's initial hematocrit. As set forth in the first row of the table (Cycle 1 start), the initial target volume for the plasma product to be collected is 889 mL, which is the same as indicated by the table of FIG. 9 for a donor having a weight of 175 lbs. and up and a hematocrit of 44 in order to harvest the FDA limit of 800 mL of raw plasma from the donor.

During each collection phase, 500 mL of whole blood may be drawn from the donor, to which anticoagulant is added at a predetermined ratio (i.e., 1:16), such that 31 mL is added for each collection cycle of 500 mL. The whole blood plus anticoagulant is separated into a plasma fraction and a red blood cell fraction.

During the first return phase (Cycle 1 return end), the red blood cells and “non-RBC” blood components are returned to the donor, so that at the end of the first return cycle the donor's hematocrit has increased to 45.6%, as calculated by the controller based on a blood volume being decreased by the amount of raw plasma collected, while the quantity of red blood cells in the total blood volume remains the same as at the start of the procedure. The controller can also account for the volume of anticoagulant that is reinfused in each return phase along with the red blood cells, as well as the residual anticoagulant in the donor's whole blood being drawn in cycles 2 and following, when determining the new hematocrit value for the next cycle. The volume of raw plasma and the total target volume of plasma product to be collected for the procedure are then recalculated based on the donor's new, increased hematocrit and raw plasma volume. This provides for a new total target collection volume of 891 mL.

The second collection phase is then performed, resulting in a total of 430 mL of plasma product comprising 386 mL of raw plasma being collected over the first two collection phases (Cycle 2 draw end). The red blood cells and “non-RBC” blood components are again returned to the donor, after which the donor's hematocrit is calculated to be 47.2%.

Two more collection phases of 500 mL are performed, each followed by a return phase, in which new values for the volume of raw plasma and total volume of plasma product to be collected are determined before the start of each collection phase. With the increasing hematocrit of the donor, the recalculated target collection volume for procedure increases to 893 mL (for the third collection phase) and then to 894 mL (for the fourth collection phase). A fifth “mini” collection cycle is performed to bring the volume of raw plasma collected up to the 800 mL permitted by the FDA nomogram for the hypothetical donor. The recalculated target collection volume of plasma product for the fifth collection phase remains at 894 mL.

Thus, as illustrated in the example above, when the target collection volume for the plasma product is recalculated for each collection phase, a target collection volume for the plasma product of 894 mL is obtained, which is required in order to collect the target volume of raw plasma of 800 mL. In contrast, 889 mL of plasma product would have been collected if the target collection volume is determined based only on the donor's initial hematocrit, or 880 mL if the target collection volume is based on the simplified FDA nomogram. In both cases, less than the target volume of 800 mL would have been collected.

The greater the accuracy with which the hematocrit of the donor can be determined, both before and during the procedure, the more likely the target volume of plasma product collected will include the maximum volume of raw plasma that can be collected for a particular donor. As described above, the hematocrit of the donor during the procedure is based on the assumptions that 100% of the red blood cells that are withdrawn in each draw cycle are reinfused in each return cycle, along with 100% of the non-RBC cellular products and a volume of anticoagulant. However, it has been determined that during the course of a blood separation procedure, interstitial fluid can shift to the intravascular space, resulting in restoring half of the withdrawn volume. See, Saito et al., Interstitial fluid shifts to plasma compartment during blood donation, Transfusion 2013; 53(11):2744-50. The shifted interstitial fluid is in addition to the red blood cells, non-RBC cellular products, and anticoagulant that are reinfused in each return phase. Thus, accounting for the shift of interstitial fluid would result in a more accurate hematocrit determination, and thus a more accurate determination of the target volume for plasma product that will result in the maximum amount of raw plasma.

The shift of interstitial fluid during plasmapheresis has been substantiated by tracking the level of Immunoglobulin G (IgG) of a donor over the course of a plasmapheresis procedure. See, e.g., Burkhardt et al., Immunoglobulin G levels during collection of large volume plasma for fractionation; Transfusion 2017; 56:417-420. If there were no shifting of interstitial fluid, the IgG level of the donor would be stable over the course of the plasmapheresis procedure. However, the IgG level has been shown to drop, and the amount that the IgG level drops is a function of the volume of interstitial fluid that has shifted to the blood system.

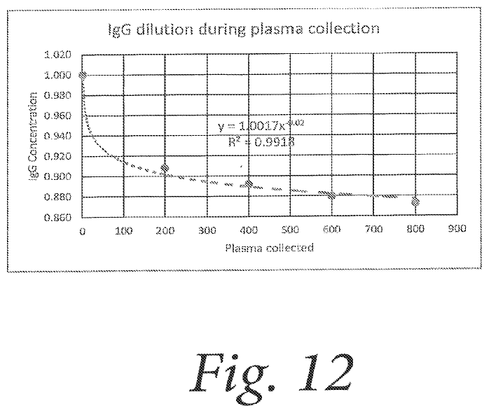

With reference to FIG. 12, a plot of volume of plasma collected (along the X-axis versus IgG concentration (along the Y-axis) that was developed empirically is shown. A 9% drop of the donor's IgG is seen from the baseline of zero plasma collected (at the start of the procedure) to 200 mL of plasma collected, and a drop of an additional 4% from 200 mL to 800 mL collected. This was attributable to a shift of interstitial fluid equal to approximately 9% of the donor's initial total blood volume (after 200 mL of plasma being collected) to approximately 13% of the donor's initial total blood volume (after 800 mL of plasma being collected).

The following relationship between the amount that the donor's IgG concentration and the volume of plasma collected has been established: y=1.0017x·^{0.02}, where y=IgG concentration and x=plasma volume collected. Thus, the percentage of the donor's blood volume that is replaced by the shift of interstitial fluid is equal to V_{b}(1−y), where V_{b} is the donor's initial volume of whole blood. Thus, the shifted volume of interstitial fluid can be calculated based on the volume of plasma collected, and this amount can be added to the volume of red blood cells, non-RBC cellular products and anticoagulant reinfused in each return phase to determine the current total blood volume, and thus hematocrit, of the donor. As can be appreciated, the controller can be configured to automatically determine the volume of interstitial fluid that has shifted based on the volume of plasma collected, and to include the shifted volume when determining the donor's hematocrit prior to each draw phase.

Alternatively, other methods that directly measure the donor's hematocrit may be employed, such as an optical sensor or, if a centrifugal separator is being used, measuring the volume of red blood cells in the centrifuge.

In some embodiments, changes over time in donor-specific parameters (e.g., weight, hematocrit, etc.) may be used to adjust a target volume of plasma product and/or raw plasma collected for that donor. Accounting for changes may, in some embodiments, improve donor safety and/or product consistency. In one example, a change in donor hematocrit from one donation to another may be used as a surrogate marker for changes in hydration status of that particular donor over time (e.g., across different encounters at the donation center). The controller may be configured to recalculate or adjust a target volume of plasma product and/or raw plasma calculated at a prior donation event based on a change in donor weight, hematocrit, etc. at a subsequent donation event.

In addition, anticoagulant may be introduced into the disposable kit prior to the commencement of the plasmapheresis procedure in pre-processing steps, such as for priming the disposable kit, performing one or more pre-cycles, or for performing other pre-procedure steps. To the extent that anticoagulant used for these purposes is ultimately directed to the plasma product collection container, it may be accounted for in determining the volume contained in the plasma collection container that results in the target volume of raw plasma being collected. This may be done, for example, by measuring the weight of the “full” container of anticoagulant and the weight of the container of anticoagulant prior to the commencement of the first draw cycle and adding that volume of anticoagulant to the target volume of plasma product. The controller can be configured to automatically perform the steps necessary to account for the anticoagulant introduced into the plasma collection container separately from the anticoagulated plasma.

In another embodiment, systems and methods of collecting plasma based on donor extracellular fluid volume (ECFV) will be described. Extracellular fluid refers to body fluid outside the cells of the donor. In some cases, extracellular fluid makes up about one third of body fluid and the remaining two thirds of body fluid may be intracellular fluid. Extracellular fluid may comprise interstitial fluid, blood plasma, lymph, and transcellular fluids (e.g., cerebrospinal fluid, fluid in the gastrointestinal tract, etc.).

In some embodiments, setting a target plasma volume to collect to be a percentage of donor extracellular fluid volume may improve the consistency of risk across different donors. Significant donor hypotensive adverse events may correlate with relative ECFV removed.

In some embodiments, setting a target plasma volume to collect to be a percentage of donor extracellular fluid volume may improve the consistency of the collected plasma product.

In some embodiments, setting a target plasma volume to collect based on donor extracellular fluid volume may provide incremental plasma at a comparable significant hypotensive adverse event (SHAE) rate to other methods of setting a target plasma volume to collect.

In some embodiments, setting a target plasma volume to collect may be more relevant considering the rapid transport of interstitial water in response to reduction in plasma volume during a donation.

The vascular system comprises both extracellular fluid (e.g., plasma) and intracellular fluid (within red blood cells). Extracellular fluid volume (ECFV) comprises (1) extravascular fluid volume (ISFV), which in some cases is about 20% of body mass and (2) intravascular fluid volume, plasma (PV), which in some cases is about 4% of body mass. Thus, ECFV can be estimated:

$$ECFV ( 24 \% of BM ) = ISFV ( 20 \% of BM ) + PV ( 4 \% of BM ) \qquad [ 9 ]$$

Extracellular fluid amount can be estimated or calculated in any of a number of different ways. In one example, extracellular fluid volume (ECFV) of the donor may be based on the weight (BM) and height (H) of the donor such that:

$$ECFV ( L ) = 0.02154 \times {BM}^{0.6469} ( kg ) \times {H}^{0.7236} ( cm ) \qquad [ 10 ]$$

where ECFV is expressed in Liters, BM is expressed in kilograms and H is expressed in centimeters. Bird et al., Indexing Glomerular Filtration Rate to Suit Children, J Nucl Med 2003; 44:1037-1043 (Bird 2003). An alternative expression in SI units is:

$$ECFV ( L ) = 0.6032 \times {BM}^{0.6469} ( kg ) \times {H}^{0.7236} ( m ) \qquad [ 11 ]$$

where H is expressed in meters.

In another example, extracellular fluid volume (ECFV) of the donor may be based on the weight (W) and height (H) of the donor such that:

$$ECFV ( L ) = {aW}^{b} {H}^{c} \qquad [ 12 ]$$

where a=0.0399, b=0.6065, and c=0.6217 for females and a=0.0755, b=0.6185 and c=0.4982 for males, and where weight W is expressed in kilograms and height H is expressed in centimeters. Bird, N. J. and Peters, A. M., New gender-specific formulae for estimating extracellular fluid volume from height and weight in adults, Nuclear Medicine Communications, 42:58-62 (2021)(Bird 2021).

In another example, extracellular fluid volume (ECFV) of the donor may be based on the weight (BM) and height (H) of the donor such that:

$$ECFV ( L ) = sqrt ( BM ( kg ) ) \times H ( m ) \qquad [ 13 ]$$

where ECFV is expressed in Liters, BM is expressed in kilograms and H is expressed in meters. Abraham et al., Extracellular Volume and Glomerular Filtration Rate in Children with Chronic Kidney Disease, Clin J Am Soc Nephrol, 2011 April; 6(4): 741-747. In another example, extracellular fluid volume of the donor may be based on the weight (BM) and height (HT) with a coefficient, such that:

$$ECFV ( L ) = 0.96 \times {BM}^{0.51} \times {H}^{0.96} \qquad [ 14 ]$$

In another example, extracellular fluid amount can be calculated as extracellular fluid weight (ECFW) based on the weight (BM) of the donor such that:

$$ECFW ( kg ) = 0.135 \times BM ( kg ) + 7.35 kg \qquad [ 15 ]$$

where ECFW and BM are expressed in kilograms.

In another example, extracellular fluid volume (ECFV) of the donor may be based on the weight (BM) and height (H) of the donor such that:

$$ECFV ( L ) = 0.0682 \times {BM}^{0.4} ( kg ) \times {H}^{0.6333} ( cm ) \qquad [ 16 ]$$

where ECFV is expressed in Liters, BM is expressed in kilograms and H is expressed in centimeters. Friis-Hansen, Body Water Compartments in Children: Changes During Growth and Related Changes in Body Composition, Pediatrics; vol. 28 no. 2, August 1961. In another example from Friis-Hansen, extracellular fluid volume (ECFV) of the donor may be based on weight alone (BM) such that:

$$ECFV ( L ) = 0.583 \times {BM}^{0.678} \qquad [ 17 ]$$

where ECFV is expressed in Liters and BM is expressed in kilograms.

In other examples, extracellular fluid amount may be calculated by first calculating total body fluid (TBF) amount using any known algorithm, calculating intracellular fluid (ICF) amount and subtracting ICF from TBF. Other calculations and/or estimations of extracellular body fluid are contemplated.

In any of the equations for calculating or estimating extracellular fluid amount, the weight used in the equation may be lean body weight instead of total body weight, along with an appropriate constant. Lean body weight may be the difference between total body weight and body fat weight. Lean body weight may be calculated from height and total body weight using formulas by Boer, James or Hume, etc. For example, the Boer formula is:

$$For males : eLBM = 0.407 W + 0.267 H - 19.2 \qquad [ 18 ]$$

$$For females : eLBM = 0.252 W + 0.473 H - 48.3 .$$

Once extracellular fluid amount (volume or weight) is calculated or estimated using one of the above equations (or a different equation), a percentage of extracellular fluid amount may be calculated as a target raw plasma amount to be collected. For example, a control circuit may be configured to calculate a target raw plasma volume to collect as a percentage of the extracellular volume, the percentage being preferably between about 4% and about 7%, between about 5% and about 6%, at least about 5%, less than about 5.5%, about 5.6% or about 5.25%. The control circuit may further be programmed to set the target raw plasma amount to collect based on a maximum plasma volume or weight (e.g., no more than 1000 mL). In some embodiments, extracellular fluid amount may be the primary parameter used to calculate the target raw plasma amount to collect, while other parameters or equations may be used to confirm the calculated amount is within a range of acceptable amounts.

In some embodiments, a controller may be programmed to calculate a target raw plasma volume to collect using a plurality of different equations, such as at least two different equations, at least three different equations, etc. A final target raw plasma volume to collect may be based on an average of the target raw plasma volumes from the different equations.

In some embodiments, ECFV may be calculated or estimated from extracellular fluid weight.

In one embodiment, a system for collecting plasma may comprise a separator configured to separate the whole blood into a plasma product and a second blood component comprising red blood cells, the blood separator having a plasma output port coupled to a plasma line configured to send the plasma product to a plasma product collection container. The system may comprise a donor line to introduce the whole blood from the donor to the separator, flow through the donor line being controlled by a first pump. The system may comprise an anticoagulant line coupled to an anticoagulant source, flow through the anticoagulant line being controlled by a second pump to combine anticoagulant with the whole blood from the donor based on an anticoagulant ratio. The system may comprise a touchscreen configured to receive input from an operator and a control circuit configured to control operation of the system. The control circuit may comprise one or more digital and/or analog circuit components configured or programmed by way of an algorithm to perform one or more of the aspects described herein. The control circuit may be coupled to the touchscreen and configured to receive at least a weight of a donor, and optionally a weight and a height of the donor. The control circuit may be configured to calculate an extracellular fluid amount (volume or weight) of the donor based at least on the weight of the donor. The control circuit may be configured to calculate a target volume for plasma product and/or raw plasma based at least in part on the extracellular fluid amount, for example, by calculating a numerical percentage or portion of the extracellular fluid amount as the target amount for plasma product or raw plasma. The control circuit may be configured to control the system to operate draw and return phases to withdraw whole blood from a donor and separate the whole blood into the plasma product and the second blood component and to return the second blood component to the donor. The second blood component may be returned to the donor during each return phase. The control circuit may further be configured to operate the draw and return phases until an amount of plasma product in the collection container equals the target amount for plasma product and/or raw plasma.

The control circuit may be configured to receive the weight and a height of the donor over a network from a donor management system, wherein the donor management system is used for qualification screening.

A target amount for plasma product and/or raw plasma may be calculated prior to initiating blood collection from the donor.

The control circuit may be configured to determine the amount of raw plasma in the collection container meets the target amount for raw plasma by weighing an amount of plasma product in the collection container and calculating the volume of raw plasma in the collection container based at least on the weight of plasma product and a percentage of anticoagulant in the collection container.

The anticoagulant ratio may be equal to a volume of whole blood divided by a volume of anticoagulant. The anticoagulant ratio may be equal to (a volume of whole blood plus a volume of anticoagulant) divided by a volume of anticoagulant. The anticoagulant ratio may be expressed as the inverse of these equations (e.g., 16 parts whole blood to 1 part AC or 1 part AC to 16 parts whole blood).

The control circuit may be configured to perform the draw and return cycles at least three times and the control circuit may be configured to determine a volume of whole blood to be drawn in a final draw phase which is different than (e.g., less than) a volume drawn in a prior draw phase.

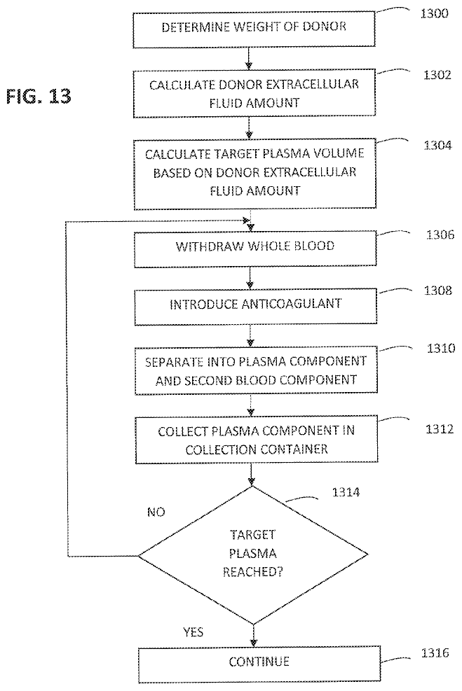

Referring now to FIG. 13, a method of collecting plasma using a plasma collection system such as that described above will be described. At block 1300, the weight of the donor is determined, for example by receiving the weight at a touchscreen input device connected to the system or by receiving the weight over a network from a donor management system or other remote computer used for donor screening. Donor height and/or other donor parameters may similarly be determined at block 1300.

At block 1302, the method comprises calculating a donor extracellular fluid amount based, at least in part, on the weight of the donor and, in alternative embodiments, based on the height of the donor. Any of the equations described above may be used, alone or together, or other equations designed to estimate extracellular fluid amount may be used. The calculation may be an estimation or approximation of donor extracellular fluid.

At a block 1304, the method comprises calculating a target plasma volume to collect based, at least in part, on a calculated percentage of donor extracellular fluid amount. The percentage may be about 5.2%, about 5.4%, about 5.6%, less than about 6.0%, more than about 5.0%, more than about 4%, or other percentages.

At block 1306, the method comprises initiating withdrawal of whole blood from the donor through a venous-access device and a first line, the first line connected to a blood component separation device. At block 1308, the method comprises introducing anticoagulant into the withdrawn whole blood through an anticoagulant line. At a block 1310, the method comprises separating the withdrawn whole blood into a plasma component and at least a second blood component (e.g., comprising red blood cells). A centrifuge, spinning membrane, or other separator may be used. At a block 1312, the method comprises collecting the plasma component from the blood component separation device and into a plasma collection container. In some embodiments, plasma collection may occur simultaneously with separation in block 1310 such that plasma is separated and continuously deposited into the collection container during the separation process.

At block 1314, the method determines whether target plasma amount is reached. This may be a target plasma product or a target raw plasma. The determination may be made using volume or weight of either plasma product or raw plasma calculated from one or more of a weigh scale, pump rotation, flow sensors, estimations, etc. If target plasma is not reached, the method continues steps 1306 through 1312 until the target is met or reached. At block 1316, the method continues to perform other steps such as presenting a report of the plasma donation process on the touchscreen or other post-processing steps.

An analysis was conducted to evaluate the incremental plasma volume and SHAE rate for a nomogram based on extracellular fluid volume (ECFV). The nomogram used was:

$${V}_{pp} = min ( ϕ ECFV , {V}_{pp . max} ) \qquad [ 19 ]$$

where V_{pp.max}=1050 mL and ϕ was to be determined. The equation from Bird 2003 was used:

$$ECFV = {aW}^{b} {H}^{c} \qquad [ 20 ]$$

where a=0.02154, b=0.6469 and c=0.7236. A database of a donor population was used to analyze the amount of plasma volume that could be obtained using the equation from Bird 2003.

An estimate for ϕ was calculated by taking the average plasma volume for this donor set using a previous nomogram (760 mL) and dividing it by the average ECFV of the donor set calculated using Bird 2003 (16287 mL). Thus,

$$ϕ = 760 / 1287 = 0.047 \qquad [ 21 ]$$

Incremental plasma volume over that obtained with a previous nomogram was calculated for the donor set with ϕ=0.0500 and ϕ=0.0525, for each gender separately and for genders combined. The modeled incremental plasma volume using the ECFV nomogram with ϕ=0.0525 was 84.6 mL overall, which closely matched the modeled incremental plasma volume using a donor blood volume nomogram of 86.9 mL (viz, V_{PP}=min(0.16*V_{DB}, V_{PPMAX}), with V_{PPMAX}=1050 mL and V_{DB}=donor blood volume.) The modeled incremental plasma volume using the ECFV nomogram with ϕ=0.0500 was 48.7 mL overall.

SHAE rate was also modeled using a Kaplan-Meir survival analysis to model SHAE rates as a function of the percentage of blood volume removed as plasma in place of the traditional time variable. Such an analysis was used to predict SHAE rates under the ECFV nomogram with ϕ=0.0500 and ϕ=0.525 separately for female donors and male donors. Probability of a SHAE as a function of percentage of ECFV removed as plasma was charted based on the Kaplan-Meier analysis. Given the probability curves, the distributions of the percentage of ECFV removed were then generated for various nomograms. Bin-wise multiplication of the SHAE probability and the percentage of donors in each bin then produced the bin-wise contributions to the overall SHAE rate. Summing these contributions gave the overall modeled SHAE rate. For the FDA 1992 Nomogram, modeled SHAE rate was 0.189 for females, 0.048 for males and 0.119 combined, assuming equal proportions of female and male donations. Modeled SHAE rates for the total blood volume nomogram were 0.235 for females, 0.071 for males and 0.153 combined. Modeled SHAE rates for the ECFV nomogram with ϕ=0.0500 were 0.171 for females, 0.064 for males and 0.118 combined. Modeled SHAE rates for the ECFV nomogram with ϕ=0.0525 were 0.214 for females, 0.071 for males and 0.143 combined. Observed SHAE rate from the database (542 SHAEs in 488,628 procedures) were 0.184 for females, 0.048 for males and 0.111 combined.

A second, similar analysis was conducted using the Bird 2021 equation, in which ECFV is based further on gender for a populating consisting of 1872 normal adults being screened for possible kidney donations. Equation [20] was used wherein a=0.0399, b=0.6064 and c=0.6217 for females and a=0.0755, b=0.6185 and c=0.4982 for males. The database of the donor population was used to analyze ECFV distribution by gender and with genders combined. Distributions in percentage of ECFV removed as plasma for the FDA 1992 nomogram were charted. Incremental plasma volume was then obtained using a previous nomogram and the ECFV nomogram using the Bird 2021 equation for ϕ=0.052, ϕ=0.054 and ϕ=0.056, for each gender separately and for genders combined. The modeled incremental plasma volume in mL using the ECFV nomogram with ϕ=0.052 was −20.0 for females, 82.6 for males and 31.4 overall. The modeled incremental plasma volume using the ECFV nomogram with ϕ=0.054 was 7.20 for females, 111.5 for males and 59.5 overall. The modeled incremental plasma volume using the ECFV nomogram with ϕ=0.056 was 34.1 for females, 138.3 for males and 86.3 overall, which closely matched the modeled incremental plasma volume using the donor blood volume nomogram of 86.9 mL.

In this second analysis, the percentages of donations which were predicted to increase or decrease pure plasma donation volumes relative to that of a prior nomogram were calculated. For the ECFV nomogram using Bird 2021 and ϕ=0.052, 29.15% of females, 81.26% of males and 55.26% combined were expected to increase pure plasma volume donations. For the ECFV nomogram using Bird 2021 and ϕ=0.054, 43.95% of females, 94.63% of males and 69.33% combined were expected to increase pure plasma volume donations. For the ECFV nomogram using Bird 2021 and ϕ=0.056, 60.59% of females, 99.21% of males and 79.94% combined were expected to increase pure plasma volume donations.

SHAE rate was also modeled using a Kaplan-Meir survival analysis to model SHAE rates as a function of the percentage of blood volume removed as plasma in place of the traditional time variable. Such an analysis was used to predict SHAE rates under the ECFV nomogram using Bird 2021 and with ϕ=0.052, ϕ=0.054 and ϕ=0.56 separately for female donors and male donors. Probability of a SHAE as a function of percentage of ECFV removed as plasma was charted based on the Kaplan-Meier analysis. Given the probability curves, the distributions of the percentage of ECFV removed were then generated for various nomograms. Bin-wise multiplication of the SHAE probability and the percentage of donors in each bin then produced the bin-wise contributions to the overall SHAE rate. Summing these contributions gave the overall modeled SHAE rate. For the FDA 1992 Nomogram, modeled SHAE rate was 0.189 for females, 0.048 for males and 0.119 combined, assuming equal proportions of female and male donations. Modeled SHAE rates for the previous total blood volume nomogram were 0.240 for females, 0.072 for males and 0.156 combined. Modeled SHAE rates for the ECFV nomogram with ϕ=0.052 were 0.133 for females, 0.064 for males and 0.098 combined. Modeled SHAE rates for the ECFV nomogram with ϕ=0.054 were 0.170 for females, 0.073 for males and 0.122 combined. Modeled SHAE rates for the ECFV nomogram with ϕ=0.056 were 0.208 for females, 0.073 for males and 0.141 combined. Observed SHAE rate from the database (542 SHAEs in 488,628 procedures) were 0.184 for females, 0.048 for males and 0.111 combined.

The second analysis using the Bird 2021 formula predicted slightly lower ECFV values; thus, higher % ECFVs were used to obtain a desired incremental plasma volume of about 85 mL. The equation

Vpp=min(0.056*ECFV,1050)) met that objective.

In a third analysis, the percentage difference in ECFV values (PECFV) was calculated between the Bird 2021 formula for females (ECFV_F) and that for males (ECFV_M) across the entire donor population of the donor database using donor weights and heights regardless of gender. Thus,

$$PECFV = 100 * ( ECFV\_F - ECFV\_M ) / ECFV\_M \qquad [ 22 ]$$

A histogram was generated. In all cases, the female formula produced lower ECFV values than the male formula by about 5%. Thus, if for any particular donor irrespective of gender we choose ECFV as

$$ECFV = ECFV\_F , \qquad [ 23 ]$$

we choose a slightly lower ECVF and a slightly lower (more conservative) pure plasma volume without reference to the donor's actual gender as input. This equation may be used when donor gender is not determined, or in other embodiments.

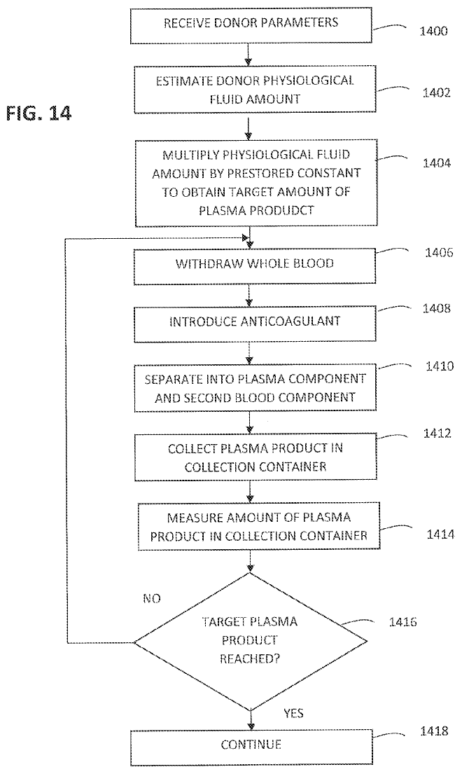

Referring now to FIG. 14, a system and method for collecting plasma according to additional embodiments will be described. Any of the structures and features described above may be used with these additional embodiments, such as venipuncture needle, separator, donor lines, anticoagulant line, calculations, etc. An input device may be configured to receive input data from an operator (e.g., via a touchscreen, nearby smartphone, etc.). The input device may be configured to receive input data over a network from a remote computer, such as a server computer configured to access and/or store donor data for a plurality of donors (e.g., donor ID, donor weight, donor height, donation history, digital image of donor, etc.).

At a block 1400, a control circuit of a reusable hardware component (e.g., plasmapheresis machine, apheresis machine, etc.) is configured to receive donor parameters from one or more input devices, such as a touch screen, keyboard, wireless or wired network interface circuit, etc. The donor parameters may comprise one or more of donor weight, donor height, donor body mass index, donor sex or gender, donor extracellular fluid volume, donor intracellular fluid volume, donor blood volume, donor plasma volume, donor body surface area, or other parameters indicative of a characteristic, such as a physiological, biological, or other characteristic of a donor.

The control circuit may be configured to estimate (block 1402) a physiological fluid amount of the donor based at least in part on the one or more donor parameters. The estimation may be done using one or more equations, such as the Bird 2003 equation, the Bird 2021 equation, the Lemmens equation, Nadler's equations, or other equations. The physiological fluid amount may comprise one or more of a donor blood volume, donor plasma volume, donor extracellular fluid volume, donor intracellular fluid volume, donor water volume, etc. The fluid amount may be a volume, weight, or other amount.

At block 1404, the control circuit may be configured to calculate a target amount of plasma product comprising raw plasma and anticoagulant by multiplying a prestored constant by the estimated physiological fluid amount. The prestored constant may be programmed into the reusable component and/or control circuit of the system during manufacture and/or during a software or firmware update. The prestored constant may be a calculation or estimation, such as will be described hereinbelow.

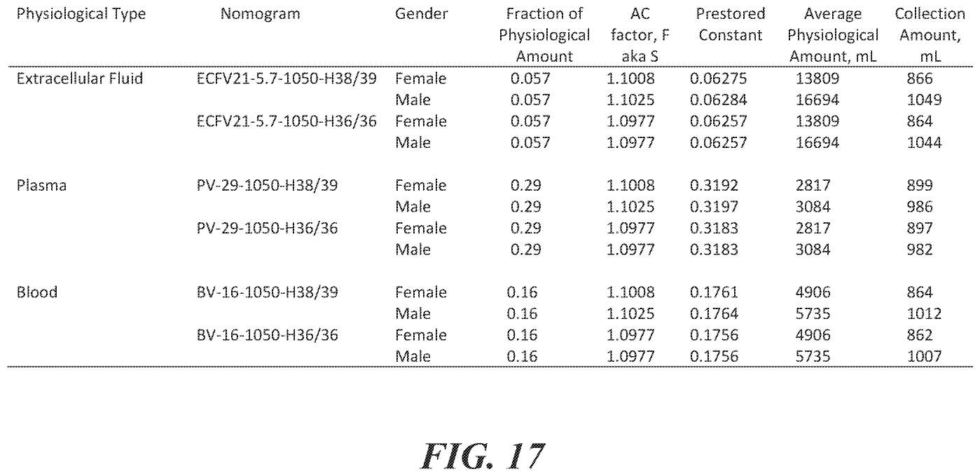

In one example, where the physiological fluid amount is extracellular fluid volume, the prestored constant may be about 0.06125 for a female donor, about 0.062715 for a male donor, at least about 0.050, less than about 0.064, and/or or other constants. In another example, where the physiological fluid amount is extracellular fluid volume, the prestored constant may be about 0.06275 for a female donor and about 0.06284 for a male donor. See also FIG. 17. In yet another example, the prestored constant may be the same for both male and female donors, for example 0.06257 where a Hct of 36 is used for both male and female donors. One or more of these constants may be used when the estimation is done using the Bird 2021 equation.

In another example, where the physiological fluid amount is a total plasma volume, the prestored constant may be about 0.285 for a female donor, about 0.3135 for a male donor, at least about 0.25, less than about 0.34, or other constants. In another example, the prestored constant may be about 0.3192 for a female donor, about 0.3197 for a male donor, at least about 0.25, less than about 0.34, and/or other constants. In yet another example, the prestored constant may be the same for both male and female donors, for example 0.3183 where a Hct of 36 is used for both male and female donors.

In another example, where the physiological fluid amount is a total blood volume in the donor, the prestored constant may be about 0.16 for a female donor, about 0.66 for a male donor, at least about 0.10, less than about 0.75, or other constants. In another example, the prestored constant may be about 0.1761 for a female donor, 0.1764 for a male donor, at least about 0.10, less than about 0.75, and/or other constants. In yet another example, the prestored constant may be the same for both male and female donors, for example 0.1756 where a Hct of 36 is used for both male and female donors.

In some embodiments, the prestored constant may be different for a male donor than for a female donor. Donor gender may be one of the donor parameters received from the input device. The control circuit may be configured to calculate the target plasma product to collect using the male prestored constant for a male donor and a female prestored constant for a female donor. Alternatively, one prestored constant may be used for both male and female donors.

At block 1406, the control circuit may be configured to control the system to operate draw and return phases to withdraw whole blood from a donor, introduce anticoagulant (block 1408), separate the whole blood into the plasma product and the second blood component (block 1410) and return the second blood component to the donor. Plasma product is collected in the collection container (block 1412). The plasma product comprises both raw plasma from the donor and anticoagulant provided from an anticoagulant source and processed through the separator.

At block 1414, the control circuit is configured to measure the amount of plasma product comprising raw plasma and anticoagulant that is collected in the container. In one embodiment, the container is weighed to determine the weight of the collected plasma product, which is then converted to a volume of plasma product. In another embodiment, other measurement devices may be used, such as optical sensors, pump rotation sensors or counters, or other devices.

At block 1416, the control circuit determines whether the measured amount of plasma product in the collection container meets the target amount of plasma product calculated in block 1404 above. This determination may be made at any of a number of stages in the collection process, for example every preset period of time (every second during collection, every five seconds, etc.), in response to certain events (e.g., an end of draw and/or return cycle, a beginning of a draw and/or return cycle), and/or at other points in time during processing. When the target plasma product comprising raw plasma and anticoagulant is reached, the process continues at block 1418 to perform other steps, such as stopping the pump, calculating and/or reporting results of the procedure, transmitting a collection report over a network to a remote computer, or other functions.

The blocks shown in FIG. 14 may be executed in the ordered sequence shown or may be executed in different orders in other embodiments. In one example, the target amount of plasma product may be calculated prior to beginning the withdrawal of whole blood. In another example, the target amount of plasma product may be calculated after the beginning of withdrawal of whole blood. In another example, the control circuit may be configured to first estimate the physiological fluid amount of the donor and then multiply a constant by the amount.

In some embodiments, donor hematocrit is not used at block 1416 or otherwise to determine when the measured amount of plasma product meets the target amount of plasma product. Instead, a constant that is not the donor's actual hematocrit may be used instead of actual hematocrit to determine when the measured amount of plasma product meets the target amount of plasma product. In other embodiments, no constant may be used to represent donor hematocrit.

In some embodiments actual donor hematocrit is not used in calculating the target amount of plasma product in block 1404 or otherwise. Instead, a constant may be used (e.g., a minimum donor Hct set by a regulatory authority, an average donor Hct over a predetermined donor population, etc.), or no constant may be used.

During a plasma collection from a donor, the total plasma product (a.k.a. total collection volume of pure or raw plasma plus anticoagulant) may be affected by the anticoagulant ratio used in the processing of the whole blood and by the donor's hematocrit. A factor F may be an anticoagulant dilution factor and may be defined:

$$F = 1 + 1 / R ( 1 - H ) \qquad [ 24 ]$$

where R is an anticoagulant ratio (such as 16:1, expressed in this equation as 16) and H is donor hematocrit expressed in this equation as a decimal, such as 0.38 to represent a 38% Hct. F can be used to determine volume of raw plasma based on volume of plasma product collected.

$$Volume of Raw Plasma = Volume of Plasma Product / F \qquad [ 25 ]$$

For example, with a Volume of Plasma Product of 900 mL, an R of 16 and an H of 50%, F=1.125 and the Volume of Raw Plasma collected would be 800 mL.

In one embodiment using the Bird 2021 equation,

$$ECFV = f ( weight , height , gender ) \qquad [ 26 ]$$

where ECFV is extracellular fluid volume, which according to the Bird 2021 equation is a function of weight, height and gender of the donor.

$$F = f ( ac\_ratio , Hct ) . \qquad [ 27 ]$$

The factor F, as shown in equation is a function of ac_ratio or R and Hct or H. Further, as shown in equation [25], Volume of Plasma Product=Volume of Raw Plasma*F. Thus:

$$Volume of Plasma Product = f ( weight , height , gender ) * x \% * F ( ac\_ratio , Hct ) . \qquad [ 28 ]$$

in the case where the target volume of raw plasma is x % of ECFV (e.g., 5.6% as described above in one example).

In another example, however, Hct can be omitted from equation and replaced with a constant C:

$$Volume of Plasma Product = f ( weight , height , gender ) * x \% * F ( ac\_ratio , C ) \qquad [ 29 ]$$

An analysis was conducted to determine values for constant C. A database of a donor population was used to analyze the plasma volume that would be obtained using equation (using actual donor Hct) vs. using equation using an average donor hematocrit of donors in the donor population as constant C in place of actual donor Hct. Also, the analysis determined the plasma volume that would be obtained using equation with the actual donor Hct vs. using equation with a minimum donor hematocrit assigned by regulatory authority as constant C in place of Hct.

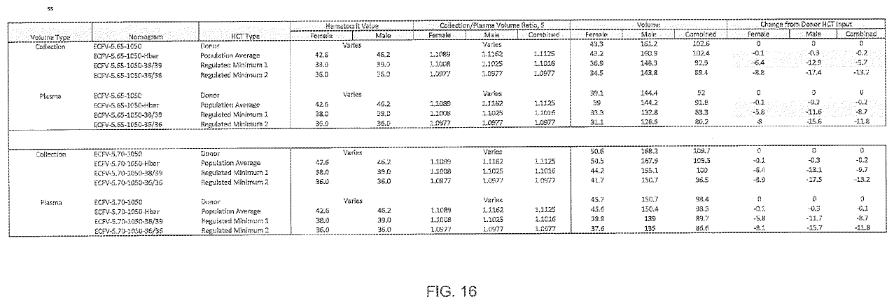

FIG. 16 is a table listing results from the analysis. Using average donor Hct (e.g., 42.6% for females and 46.2% for males) instead of actual Hct would have resulted in negligible changes in average collection volume (0.1 mL less for females and 0.3 mL less for males). Using minimum Hct instead of actual Hct would have resulted in average total collection volume of 6.4 mL less for females and 12.9 mL less for males. Average raw plasma volumes were also calculated and would have resulted in negligible changes (0.07 mL for females, 0.25 mL for males using average Hct). Average raw plasma volumes using the minimum Hct would have resulted in 5.8 mL less for females and 11.6 mL less for males. Combining genders, average total collection volume was calculated to be 0.20 less for both genders using average Hct and 9.62 mL less for both genders using minimum Hct. Raw plasma volume for combined genders was calculated to be 0.2 mL less using average Hct and 8.7 mL less using minimum Hct.

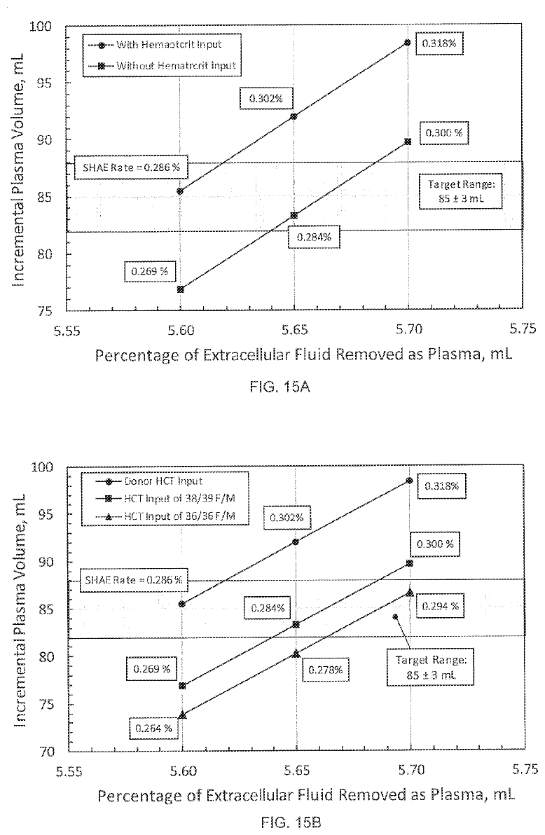

In one embodiment, the minimum Hct may be used as the constant C. In an alternative embodiment, the average Hct over a predetermined population may be used for the constant C. The average Hct of a population of donors may be initially unknown. Further, the minimum Hct is a conservative estimate assigned by regulatory authority (FDA). For lower Hct donors, using the average Hct will collect more plasma than using the actual Hct. Selecting the minimum Hct as the constant C is a more conservative estimate for optimizing donor safety. According to the analysis, using the minimum Hct as the constant C will result in collecting about 10 mL less plasma than using the actual donor Hct, on average. To accommodate for the 10 mL difference, it was proposed to increase the x % in equation to recover at least a portion of the 10 mL. To determine the amount to increase the x %, an analysis of incremental plasma volume achieved over the 1992 FDA nomogram was conducted on the population of donors using the actual Hct of the donor and the minimum Hct. The results were plotted based on percentage of extracellular fluid removed as plasma in mL and are shown in the attached FIG. 15A. For a desired target range of incremental plasma volume over the 1992 FDA nomogram of 85+/−3 mL, a percentage of 5.65% may be selected as fitting within the target range when using the minimum Hct in place of actual donor Hct. In other words, a percentage of 5.60% of ECFV that met the target range when using actual donor Hct was increased by 0.05% to 5.65% when using the minimum Hct, in order to maintain incremental plasma over the previous 1992 FDA nomogram within the target range of 85+/−3 mL. SHAE rates were also calculated for several data points, with the SHAE rate of the 5.65 point being estimated to be between 0.269% and 0.300%.

Referring to FIG. 15B, to determine the amount to increase the x %, an analysis of incremental plasma volume achieved over the 1992 FDA nomogram was conducted on the population of donors using the actual Hct of the donor and Hct inputs of 38 for females/39 for males and 36 for females/36 for males. The results were plotted based on percentage of extracellular fluid removed as plasma in mL and are shown in the attached FIG. 15B. For a desired target range of incremental plasma volume over the 1992 FDA nomogram of 85+/−3 milliliters, a percentage of 5.70% was selected as fitting within the target range when using the Hct input of 36 for females and 36 for males in place of actual donor Hct. In other words, a percentage of 5.60% of ECFV that met the target range when using actual donor Hct was increased by 0.10% to 5.70% when using 36 Hct, in order to maintain incremental plasma over the previous 1992 FDA nomogram within the target range of 85+/−3 mL. SHAE rates were also calculated for several data points, with the SHAE rate of the 5.70% point being estimated to be 0.294%.

In various embodiments, with reference to FIG. 17, the fraction of ECFV may be selected from values of at least about 0.055% and/or less than about 0.0575%. The fraction of total plasma volume may be at least about 0.25 and/or less than about 0.35. The fraction of total blood volume may be at least about 0.12 and/or less than about 0.24.

In one embodiment, equation above can be expressed as follows:

$$Volume of Plasma Product = f ( weight , height , gender ) * FH ( x \% , ac\_ratio , C ) \qquad [ 29.1 ]$$

Where FH is a prestored constant that is a function of the percentage of ECFV to collect and the other variables as previously defined. Where ac_ratio is 16, constant C is set to a minimum Hct of 39 for male donors and 38 for female donors (as set per regulatory authority), and x % is set to the increased percentage of 5.65%, the equation becomes:

$$Volume of Plasma Product = f ( weight , height , gender ) * FH ( constant ) \qquad [ 30 ]$$

$$Volume of Plasma {Product}_{f} = ECFV * 0.6125 ( female ) \qquad [ 31 ]$$

$$Volume of Plasma {Product}_{m} = ECFV * 0.62715 ( male ) \qquad [ 32 ]$$

In this case, the prestored constant used in the method shown in FIG. 14 above is 0.6125 for a female donor and 0.62715 for a male donor. In alternate embodiments, a single constant may be used for both genders, which may be an average of the two constants or the lower of the two constants, or another constant.

In an alternate embodiment, and with reference to FIG. 17, x % may be set to the increased percentage of 5.70% with Hct set to 39 for male donors and 38 for female donors (see Nomogram ECFV21-5.7-1050-H38/39). The Fraction of Physiological Amount (% of ECFV taken as target plasma product) is 0.057 or 5.70%. The AC factor F in equation is 1.1008 for female donors and 1.1025 for male donors. The prestored constant C is then 0.06275 for female donors and 0.06284 for male donors, leading to a collection amount of 866 mL for females and 1049 mL for males.

According to another embodiment, and with reference to FIG. 17, Hct may be set to a constant of 36 for both male and female donors (see Nomogram ECFV21-5.70-1050-H36/36). The Fraction of Physiological Amount (% of ECFV taken as target plasma product) is 0.057 or 5.70%. The AC factor F in equation [27] is 1.0977. The prestored constant C is then 0.06257 for both male and female donors, leading to a collection amount of 864 mL for females and 1044 mL for males.

According to one advantage, using the prestored constant—as described in the analysis above and in the method of FIG. 14—has the advantage of being able to proceed without needing actual donor Hct. As between donor weight, height and Hct, the donor Hct has the most potential error due to how it is determined for each donor. Assigning a fixed Hct as the minimum Hct (e.g., 39 for men and 38 for women) provides the advantage of a conservative approach, which provides safer estimates for collecting plasma. Further, the calculation of target plasma product may be easier, may require less processing power and time to thereby improve the operation of the control circuit, and/or may be less prone to error because the prestored constant is multiplied by the estimated ECFV for the donor. Target plasma product may be 10.0% more for female donors and 10.2% more for male donors as compared to a prior nomogram, for example increasing target plasma product from 1000 mL to 1100 mL for female donors and 1000 mL to 1102 mL for male donors.

The analysis conducted demonstrated that the plasma collection algorithm can calculate a target plasma product to collect without using actual donor Hct in the calculation of donor physiological fluid and/or without using actual donor Hct in the comparison of plasma product collected to target plasma product. The analysis also demonstrated that the percentage of ECFV volume collected could be increased from 5.60% to 5.65% or 5.70% in some embodiments with acceptable SHAE rates and meeting an objective of increasing raw plasma yield by about 85 mL over the 1992 FDA nomogram.

In some embodiments, a control circuit of a plasma collection device may be configured to calculate a target plasma product comprising raw plasma and anticoagulant by multiplying an estimated physiological fluid volume of a donor by a constant.

In some embodiments, a control circuit of a plasma collection device may be configured to calculate a target plasma product comprising raw plasma and anticoagulant by multiplying an estimated physiological fluid volume of a donor by only a constant and not by any variable.

In some embodiments, a control circuit of a plasma collection device may be configured to calculate a target plasma product comprising raw plasma and anticoagulant using an estimated physiological fluid volume of a donor and a predefined constant, but not using an anticoagulant ratio in the calculation, the anticoagulant ratio having been previously provided for in the predefined constant.

In some embodiments, the prestored constant or constants may be predetermined or precalculated by a manufacturer of the plasma collection device and programmed into a memory of the plasma collection device during manufacture. The prestored constant or constants may be calculated based on one or more of a target percentage of physiological fluid volume (x %), an anticoagulant ratio (R), and a constant C used in place of an actual donor Hct, such as a minimum donor Hct per regulatory rule, an average donor Hct over a predetermined donor population, or other constant.

While a target range of improved plasma collection over a prior nomogram may have been 85+/−3 mL in the analysis conducted above, other target ranges of improved plasma collection may be used to arrive at the target percentage of physiological fluid volume, such as at least 30 mL, at least 60 mL, less than 100 mL, less than 120 mL, etc.

According to another embodiment, a method for collecting plasma may comprise receiving one or more donor parameters selected from donor weight, donor height, donor body mass index and donor sex. Any one or more of these donor parameters may be received from a user input device coupled to the plasma collection device (e.g., touch screen, keyboard, etc.) or from another input device such as a network interface circuit configured to receive the donor parameters from a remote computer, such as a donor management system used for functions such as donor screening, donor recruitment, and/or other functions. For some ECFV equations, only weight may be used. In other ECFV equations, weight and height may be used. In still other ECFV equations, weight, height and gender may be used.

The method may further comprise estimating a donor physiological fluid amount based, at least in part, on the one or more donor parameters. The estimated donor physiological fluid amount may be selected from the group comprising total blood amount, total plasma amount, extracellular fluid amount, intracellular fluid amount, interstitial fluid amount, transcellular fluid amount, lymph amount, and/or other fluid amounts expressed in volume, weight, area (e.g., body surface area) or otherwise and expressed in any of a variety of units. Any of the equations described hereinabove or otherwise available, such as Lemmens, Bird 2003, Bird 2021, etc., may be used to estimated one or more donor physiological fluid amounts. The donor physiological fluid amounts may be calculated by the plasma collection machine as part of a plasma collection procedure for a particular donor.

The method may further comprise calculating a target amount of plasma product to collect by multiplying the estimated donor physiological fluid amount by a prestored constant. A prestored constant or constants may be determined during product development and programmed into the plasma collection device during manufacture, for example into firmware or other memory. In some embodiments, the calculation may comprise only multiplying the estimated donor physiological fluid amount by the prestored constant to arrive at the target amount of plasma product to collect. In other embodiments, other factors may be used in the calculation. The prestored constant may be a constant that has been previously calculated (during product development, on a remote server computer, or even on the plasma collection device itself) based on a predetermined percent of physiological fluid volume (e.g., x %), an anticoagulant ratio (R) expressed as a ratio, fraction, decimal or otherwise, and/or a donor Hct value or constant (C) taking the place of the donor Hct value.

The prestored constant may be greater than about 0.050 and/or less than about 0.064 or about 0.06257 where the physiological fluid is ECFV.

As shown in FIG. 17, the prestored constant may be at least about 0.28 and/or less than about 0.34, or about 0.0.3183 where the physiological fluid amount is a total donor plasma volume and Hct is set to 36 for both male and female donors. When Hct is set to 38 for female donors and 39 for male donors, the prestored constant may be about 0.3192 for a female donor and about 0.3197 for a male donor.

As also shown in FIG. 17, the prestored constant may be at least about 0.10 and/or less than about 0.20, or about 0.1756 when the physiological fluid is total donor blood volume and Hct is set to 36 for both male and female donors. When Hct is set to 38 for female donors and 39 for male donors, the prestored constant may be about 0.1761 for a female donor and about 0.1764 for a male donor.

Other constants are contemplated. In various embodiments, any of the prestored constants described in FIG. 17 or otherwise described herein may be within a range of at least about 50%, at least about 75%, less than about 150%, and/or less than about 200% of the value indicated. For example, where the donor physiological fluid amount is ECFV and Hct is set at 36 for male and female donors, the prestored constant may be at least about 0.3128 (50% of the value indicated: 0.06257), at least about 0.04692 (75% of the value indicated: 0.06257, etc.), less than about 0.07508, and/or less than about 0.12514. For example, different embodiments may assume other values for Hct (at least about 33 Hct for male and/or female donors, less than about 45 Hct for male and/or female donors), other values for the AC ratio (15:1, 16:1, 17:1, etc. which may be measured as (volume AC+volume whole blood)/(volume whole blood) or (volume AC/volume whole blood)), and/or other values for the fraction of physiological fluid amount (less than about 7.5% of ECFV, more than about 4.5% of ECFV), which may lead to other predetermined constants being used based on desired collection amount, desired SHAE rates, etc.

The method may further comprise withdrawing whole blood from the donor through a venous-access device to a blood component separation device. The calculations and estimations described above may occur prior to withdrawing whole blood, during whole blood withdrawal, and/or after beginning whole blood withdrawal. The method may comprise introducing anticoagulant into the withdrawn whole blood, separating the withdrawn whole blood into a plasma product comprising raw plasma and anticoagulant and at least a second blood component (e.g., red blood cells), and collecting the plasma product from the blood component separation device into a plasma collection container.

The method may further comprise determining whether the collected plasma product (comprising raw plasma and anticoagulant) has reached the target amount of plasma product to collect. This determination may be made at various times during the processing of blood from withdrawal to mixing with anticoagulant to separation to return of red blood cells to the donor. The determination may be made by receiving a signal from a weigh scale which weighs the plasma product collection container. The determination may comprise converting a weight of the plasma product to a volume of plasma product. The determination may comprise comparing the volume of plasma product measured to the target amount of plasma product to collect. The determination may comprise determining the collected plasma product has reached the target amount of plasma product to collect when the collected plasma product equals or approximately equals the target amount of plasma product or a value derived from the target amount of plasma product.

The control circuit of the plasma collection machine may be programmed with one or more of a number of different nomograms. The nomogram may be based on percentage or fraction of donor blood volume (BV), plasma volume (PV), extracellular fluid volume (ECFV) or weight (W) removed as plasma. These nomograms may be expressed as:

$${V}_{pp} = min ( {ϕ}_{BASE , N} {V}_{BASE} , {V}_{PP , M AX , M} ) \qquad [ 33 ]$$

where V_{PP} is target volume of pure (or raw) plasma, the subscript BASE refers to the nomogram basis as indicated above (PV, BV, ECFV or W), ¢ is a fraction of the donor base volume, V_{PP, MAX, M} is a maximum pure plasma volume, and the subscripts N and M refer to particular values of ϕ and V_{PP, MAX} of interest. Exemplary equations for calculating BV and ECFV are provided hereinabove (e.g., Lemmens, Bird 2003, Bird 2021, etc.). Plasma volume may be calculated as:

$${V}_{P} = ( 1 - H ) {V}_{B} \qquad [ 34 ]$$

where H is donor Hct (or a minimum Hct or average Hct over a set of donors). Weight may be measured in kilograms.

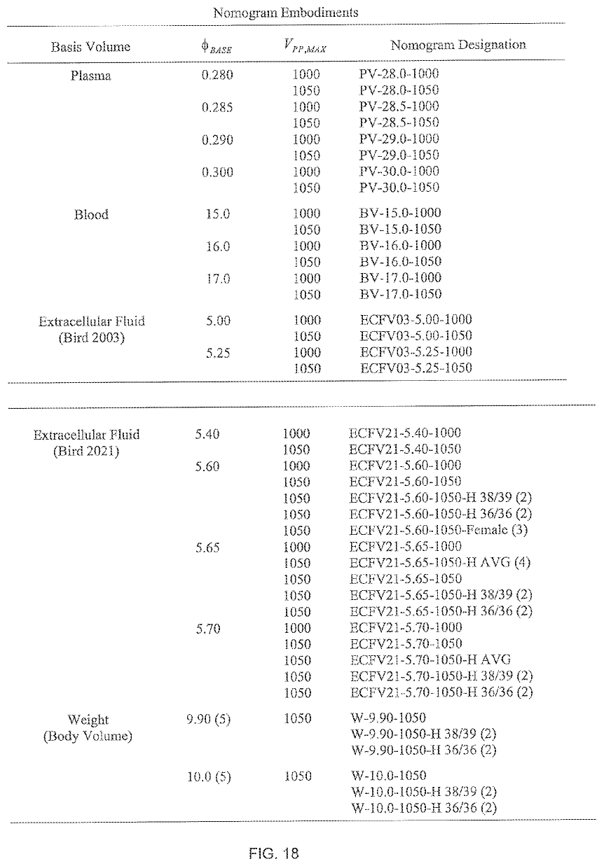

The control circuit may be programmed with any nomogram having one or more characteristics, such as a basis (PV=Plasma, BV=Blood, EDFV03=Bird 2003, ECFV21=Bird 2021, W=Weight), a percentage of basis volume removed as plasma (1000BASE), a maximum pure plasma volume (V_{PP, MAX}) and/or additional data such as an assumed Hct. The control circuit of the plasma collection system may be programmed with any one or more of the nomograms shown in FIG. 18 which may use one or more of the equations described hereinabove. For example, nomogram designation PV-28.0-1000 indicates a nomogram based on a target raw plasma of 28.0% of total donor plasma volume, not to exceed 1000 mL. Nomogram designation ECFV21-5.70-1050 indicates a nomogram based on a target pure plasma of 5.70% of donor extracellular fluid volume using the Bird 2021 equation, not to exceed a maximum of 1050 mL of pure plasma. As another example, nomogram designation ECFV21-5.70-1050-H 38/39 indicates a nomogram based on a target pure plasma of 5.70% of donor extracellular fluid volume using the Bird 2021 equation, not to exceed a maximum of 1050 mL of pure plasma, with hematocrit taken as the minimum allowable (38% for females, 39% for males) which calculating collection volume.

Referring to FIG. 18, for the nomograms designated as −H 38/39, the hematocrit may be taken as the minimum allowable (38% for females and 39% for males). For those designated −H 36/36, a fixed hematocrit of 36% may be used for both genders. V_{PP} may first be evaluated using the nomogram. Then the collection volume (pure plasma plus anticoagulant) may be calculated assuming these fixed hematocrits. Referring again to FIG. 18, the nomogram indicated as ECFV21-5.60-1050-Female (3) may use the Bird 2021 equation for females for all donors, male and female. Nomograms with the designation −H AVG may use the average hematocrits for female and males to calculate the collection volume (pure plasma plus anticoagulant), which for one set of donors may be 42.6% for males and 46.2% for females.

The original 1992 nomogram and the optimized nomogram are based on donor weight applied in a stepwise fashion. The nomograms described as Weight (Body Volume) in FIG. 18 use weight continuously in accordance with Equation under the assumption that the body density is approximately 1.0 g/mL. ϕ_{BASE} may be expressed in milliliters per kilogram of weight (9.90 mL/kg weight). The ϕ_{BASE} may alternatively be expressed as percentages of body volume, in this case 0.99% and 1.00%. Other values for ØBASE are contemplated, such as at least about 8 mL/kg, at least about 9 ml/kg, less than about 10.5 ml/kg, less than about 12.0 ml/kg, etc.

For each of the nomograms listed in FIG. 18, V_{PP, MAX} may be at least about 1000 mL, at least about 950 mL, at least about 800 mL, less than about 1050 mL, less than about 1100 mL, less than about 1200 mL, etc. Any of the nomograms described herein may comprise an upper limit V_{PP, MAX}.

For each of the nomograms listed in FIG. 18, a target may be calculated as target pure plasma or target plasma product (pure or raw plasma plus anticoagulant), to be used as a target for completion of the plasma donation for the donor. For each of the nomograms listed in FIG. 18, calculating a target plasma product may use one or more of actual donor hematocrit, fixed donor hematocrits (e.g., 36, 38, 39), same or different fixed hematocrits for female and male donors, an average of donor hematocrits over a population of donors, or other values.

In various embodiments, the control circuit of the plasma collection device may be configured to estimate a physiological fluid amount of the donor based at least in part on one or more donor parameters and using any of the nomograms described in FIG. 18. Further, control circuit may be configured to calculate a target amount of plasma product comprising raw plasma and anticoagulant by multiplying a prestored constant by the estimated physiological fluid amount, the prestored constant being determined based on the respective nomogram described in FIG. 18.

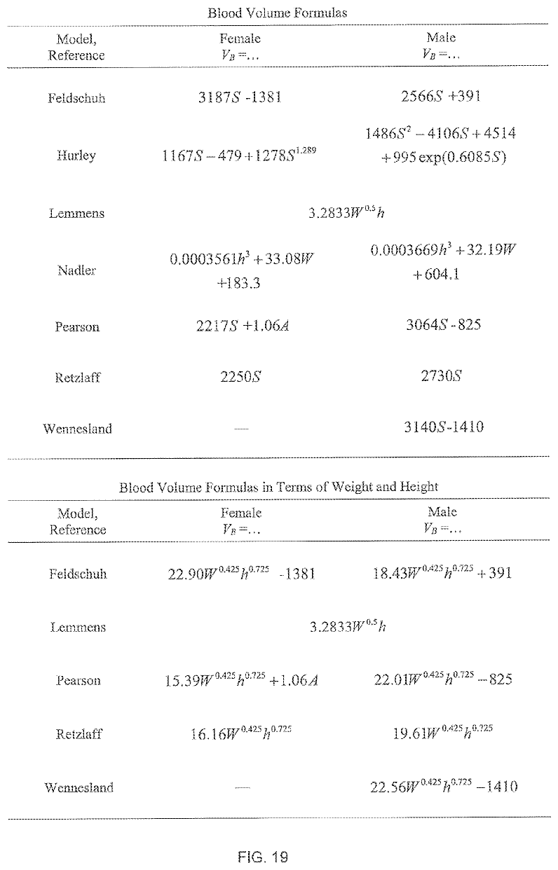

Referring now to FIG. 19, donor total blood volume (and/or total plasma volume) may be calculated or estimated using any of the models shown based on weight (W), height (h) and/or age (A). Feldschuh, J. and Y. Enson. Prediction of Normal Blood Volume: Relation of Blood Volume to Body Habitus. Circulation, 56(4): 605-612 (1977). Hurley, P. J. Red Cell and Plasma Volumes in Normal Adults. Journal of Nuclear Medicine, 16(1): 46-52 (1975). Nadler, S. B. et al. Prediction of Blood Volume in Normal Human Adults. Investigative Surgery, 51(2): 224-232 (1961). T. C. Pearson et al. Interpretation of measured red cell mass and plasma volume in adults: Expert Panel on Radionuclides of the International Council for Standardization in Haematology. British Journal of Haematology, 89: 748-756 (1995). J. A. Retzlaff et al. Erythrocyte Volume, Plasma Volume, and Lean Body Mass in Adult Men and Women. The Journal of Hematology, 33(5): 649-667 (1969). Wennesland, R. et al. Red Cell, Plasma and Blood Volume in Healthy Men by Radiochromium (Cr 51) Cell Tagging. Where used, the body surface area S is given by:

$$S = 0.0 \; 07184 {W}^{0.245} {h}^{0.725} \qquad [ 34 ]$$

where W is weight in kilograms and his height in centimeters. DuBois, D. and EF DuBois. A Formula to Estimate the Approximate Surface Area If Height and Weight Be Known. Archives of Internal Medicine, 17: 863-871 (1916). The equation for surface area (S) can be expressed as:

$$S = a {W}^{b} {h}^{c} + d \qquad [ 35 ]$$

where d=0. Thus, any model which simply scales S by some coefficient can be expressed in the same mathematical form as shown in the table at the bottom of FIG. 19 (Blood Volume Formulas in Terms of Weight and Height).

Additional embodiments of the methods and systems disclosed herein are set forth below.

In a first embodiment, a system for collecting plasma is provided. The system includes a separator configured to separate the whole blood into a plasma product and a second blood component comprising concentrated cells. The blood separator includes a plasma output port coupled to a plasma line configured to send the plasma product to a plasma product collection container. The system includes: a donor line configured to introduce the whole blood from the donor to the separator, flow through the donor line being controlled by a first pump; an anticoagulant line coupled to an anticoagulant source, flow through the anticoagulant line being controlled by a second pump to combine anticoagulant with the whole blood from the donor based on an anticoagulant ratio; an input device configured to receive input from an operator and/or over a network from a remote computer; and a control circuit configured to control operation of the system. The control circuit is coupled to the input device and configured to receive one or more donor parameters, to estimate a physiological fluid amount of the donor based at least in part on the one or more donor parameters, to calculate a target amount of plasma product comprising raw plasma and anticoagulant by multiplying a prestored constant by the estimated physiological fluid amount, the control circuit configured to control the system to operate draw and return phases to withdraw whole blood from a donor and separate the whole blood into the plasma product and the second blood component and to return the second blood component to the donor, wherein the control circuit is further configured to operate the draw and return phases until a measured amount of plasma product in the collection container meets the target amount of plasma product.

A second embodiment includes the system of the first embodiment wherein the control circuit is configured to not use donor hematocrit in determining when the measured amount of plasma product meets the target amount of plasma product.

A third embodiment includes the system of the first embodiment wherein the control circuit is configured to not use donor hematocrit in calculating the target amount of plasma product.

A fourth embodiment includes the system of the first embodiment wherein the estimated physiological fluid amount is an extracellular fluid volume.

A fifth embodiment includes the system of the fourth embodiment, wherein the prestored constant is between about 0.050 and about 0.064.

A sixth embodiment includes the system of the fourth embodiment, wherein the control circuit is configured to calculate the extracellular fluid volume of the donor based on the weight (W), height (H), and sex of the donor such that ECFV=0.0399×W^{0.6065}×H^{0.6217} for females and ECFV=0.0755×W^{0.6185}×H^{0.4982} for males.

A seventh embodiment includes the system of the first embodiment, wherein the estimated physiological fluid amount is a total plasma volume.

An eighth embodiment includes the system of the seventh embodiment, wherein the prestored constant is between about 0.25 and about 0.34.

A ninth embodiment includes the system of the first embodiment, wherein the estimated physiological fluid amount is a total blood volume.

A tenth embodiment includes the system of the ninth embodiment, wherein the prestored constant is between about 0.10 and about 0.75.

An eleventh embodiment includes the system of the first embodiment, wherein the prestored constant is different for a male donor than for a female donor, donor gender data being received from the input device and used to select from a male prestored constant and a female prestored constant.

A twelfth embodiment includes the system of the first embodiment, wherein a weight and a height of the donor are received over a network from a donor management system, wherein the donor management system is used for qualification screening.

A thirteenth embodiment includes the system of the first embodiment, wherein the target amount for plasma product is calculated prior to initiating blood collection from the donor.

A fourteenth embodiment includes the system of the first embodiment, wherein the control circuit is configured to measure the amount of plasma product comprising raw plasma and anticoagulant in the collection container using a weigh scale.

In a fifteenth embodiment a method for collecting plasma is provided. The method includes (a) receiving one or more donor parameters selected from donor weight, donor height, donor body mass index and donor sex; (b) estimating a donor physiological fluid amount based, at least in part, on the one or more donor parameters, wherein the estimated donor physiological fluid amount is selected from total blood volume, total plasma volume and extracellular fluid volume; (c) calculating a target amount of plasma product to collect, the target amount of plasma product comprising raw plasma and anticoagulant, the target amount of plasma product calculated by multiplying the estimated donor physiological fluid amount by a prestored constant; (d) withdrawing whole blood from the donor through a venous-access device to a blood component separation device; (e) introducing anticoagulant into the withdrawn whole blood; (f) separating the withdrawn whole blood into a plasma product comprising raw plasma and anticoagulant and at least a second blood component; (g) collecting the plasma product from the blood component separation device into a plasma collection container; (h) determining whether the collected plasma product comprising raw plasma and anticoagulant has reached the target amount of plasma product to collect; and
(i) continuing steps (d) through (h) until the collected plasma product reaches the target amount of plasma product to collect.

A sixteenth embodiment includes the method of the fifteenth embodiment wherein the estimated donor physiological fluid amount is donor extracellular fluid.

A seventeenth embodiment includes the system of the sixteenth embodiment, wherein the prestored constant is between about 0.05 and about 0.07.

An eighteenth embodiment includes the system of the seventeenth embodiment, wherein the prestored constant comprises a first constant for use with a male donor and a second constant different than the first constant for use with a female donor.

A nineteenth embodiment includes the system of the fifteenth embodiment, wherein actual donor hematocrit is not used in estimating donor physiological fluid amount, wherein actual donor hematocrit is not used in calculating a target amount of plasma product to collect.

In a twentieth embodiment a system for collecting plasma is provided. The system includes a blood separator configured to separate the whole blood into a plasma product and a second blood component comprising concentrated cells, the blood separator having a plasma output port coupled to a plasma line configured to send the plasma product to a plasma product collection container, the plasma product comprising raw plasma and anticoagulant; a donor line configured to introduce the whole blood from the donor to the blood separator, flow through the donor line being controlled by a first pump; an anticoagulant line coupled to an anticoagulant source, flow through the anticoagulant line being controlled by a second pump to combine anticoagulant with the whole blood from the donor based on an anticoagulant ratio; an input device configured to receive input from an operator; and a control circuit programmed to control operation of the system, the control circuit coupled to the input device and programmed to receive at least a donor's weight, to estimate a donor physiological fluid amount based at least in part on the donor's weight and without using the donor's actual hematocrit, to determine a target volume for plasma product comprising raw plasma and anticoagulant as a predetermined percentage of the donor's physiological fluid amount and without using the donor's actual hematocrit, to control the system to operate draw and return phases to withdraw whole blood from the donor and separate the whole blood into a the collected plasma product and the second blood component and to return the second blood component to the donor, wherein the control circuit is programmed to perform the draw and return phases at least three times, wherein the control circuit is configured to compare the amount of collected plasma product to the target volume of plasma product and to continue processing the whole blood until the amount of collected plasma product reaches the target volume of plasma product.

In a twenty-first embodiment system for collecting plasma is provided. The system includes a separator configured to separate the whole blood into a plasma product and a second blood component comprising concentrated cells, the blood separator having a plasma output port coupled to a plasma line configured to send the plasma product to a plasma product collection container; a donor line configured to introduce the whole blood to the separator, flow through the donor line being controlled by a first pump; an anticoagulant line coupled to an anticoagulant source, flow through the anticoagulant line being controlled by a second pump to combine anticoagulant with the whole blood from the donor based on an anticoagulant ratio; an input device configured to receive input from an operator; and a control circuit configured to control operation of the system, the control circuit coupled to the input device and configured to receive a weight of a donor, to calculate an extracellular fluid amount of the donor based at least on the weight of the donor, to calculate a target amount for plasma product and/or raw plasma based at least in part on the extracellular fluid amount, the control circuit configured to control the system to operate draw and return phases to withdraw whole blood from a donor and separate the whole blood into the plasma product and the second blood component and to return the second blood component to the donor, wherein the control circuit is further configured to operate the draw and return phases until a volume of plasma product in the collection container meets the target amount for plasma product and/or raw plasma.

In a twenty-second embodiment includes the system of the twenty-first embodiment wherein the control circuit is configured to receive the weight and a height of the donor over a network from a donor management system, wherein the donor management system is used for qualification screening.

A twenty-third embodiment includes the system of the twenty-first embodiment, wherein the target amount for plasma product and/or raw plasma is calculated prior to initiating blood collection from the donor.

A twenty-fourth embodiment includes the system of the twenty-first embodiment, wherein the control circuit is configured to determine an amount of raw plasma in the collection container meets the target amount for raw plasma by weighing an amount of plasma product in the collection container and calculating a volume of raw plasma in the collection container based at least on the weight of plasma product and a percentage of anticoagulant in the collection container.

A twenty-fifth embodiment includes the system of the twenty-first embodiment, wherein the control circuit is configured to calculate the extracellular fluid amount of the donor as an extracellular fluid volume (ECFV) based on the weight (W), height (H), and sex of the donor such that ECFV=0.0399×W^{0.6065}×H^{0.6217} for females and ECFV=0.0755×W^{0.6185}×H^{0.4982} for males.

A twenty-sixth embodiment includes the system of the twenty-first embodiment, wherein the control circuit is configured to calculate the target volume of plasma product and/or raw plasma by calculating a percentage of the extracellular fluid amount of the donor.

A twenty-seventh embodiment includes the system of the twenty-first embodiment, wherein the control circuit is configured to perform the draw and return cycles at least three times and the control circuit is configured to determine a volume of whole blood to be drawn in a final draw phase which is different than a volume drawn in a prior draw phase.

A twenty-eighth embodiment includes the system of the twenty-first embodiment, wherein the control circuit is configured to calculate the extracellular fluid amount of the donor as an extracellular fluid volume (ECFV) based on the weight (BM) and height (H) of the donor such that ECFV (L)=0.02154×BM^{0.6469} (kg)×H^{0.7236} (cm).

A twenty-ninth embodiment includes the system of the twenty-first embodiment, wherein the control circuit is configured to calculate the extracellular fluid amount of the donor as an extracellular fluid volume (ECFV) based on the weight (BM) and height (H) of the donor such that ECFV (L)=sqrt(BM (kg))×H (m).

A thirtieth embodiment includes the system of the twenty-first embodiment, wherein the control circuit is configured to calculate the extracellular fluid amount of the donor as an extracellular fluid weight (ECFW) based on the weight (BM) of the donor such that ECFW (kg)=0.135×BM (kg)+7.35 kg.

In a thirty-first embodiment, a method for collecting plasma is provided The method includes (a) determining the weight of a donor; (b) calculating a donor extracellular fluid amount based, at least in part, on the weight of the donor; (c) calculating a target plasma volume to collect based, at least in part, on a calculated percentage of donor extracellular fluid amount; (d) withdrawing whole blood from the donor through a venous-access device and a first line, the first line connected to a blood component separation device; (e) introducing anticoagulant into the withdrawn whole blood through an anticoagulant line; (f) separating the withdrawn whole blood into a plasma component and at least a second blood component; (g) collecting the plasma component from the blood component separation device and into a plasma collection container; (h) continuing steps (d) through (g) until the target plasma volume to collect is reached in the plasma collection container.

A thirty-second embodiment includes the method of the thirty-first embodiment wherein the target plasma volume to collect is a target raw plasma volume.

A thirty-third embodiment includes the method of the thirty-first embodiment, wherein the donor extracellular fluid amount is calculated as an extracellular fluid volume (ECFV) based on the weight (BM) and height (H) of the donor such that ECFV (L)=0.2154×BM^{0.6469} (kg)×H^{0.7236} (cm).

A thirty-fourth embodiment includes the method of the thirty-first embodiment, wherein the control circuit is configured to calculate the extracellular fluid amount of the donor as an extracellular fluid volume (ECFV) based on the weight (W), height (H), and sex of the donor such that ECFV=0.0399×W^{0.6065}×H^{0.6217} for females and ECFV=0.0755×W^{0.6185}×H^{0.4982} for males.

A thirty-fifth embodiment includes the method of the thirty-first embodiment, wherein the donor extracellular fluid amount is calculated as an extracellular fluid weight (ECFW) based on the weight (BM) of the donor such that ECFW (kg)=0.135×BM (kg)+7.35 kg.

A thirty-sixth embodiment includes a system for collecting plasma is provided. The system includes a blood separator configured to separate the whole blood into a plasma product and a second blood component comprising concentrated cells, the blood separator having a plasma output port coupled to a plasma line configured to send the plasma product to a plasma product collection container; a donor line configured to introduce the whole blood from the donor to the blood separator, flow through the donor line being controlled by a first pump; an anticoagulant line coupled to an anticoagulant source, flow through the anticoagulant line being controlled by a second pump to combine anticoagulant with the whole blood from the donor based on an anticoagulant ratio; an input device configured to receive input from an operator; and a control circuit programmed to control operation of the system, the control circuit coupled to the input device and programmed to receive at least a donor's weight, to estimate the donor's extracellular fluid amount based on the donor's weight, to determine a target volume for plasma product and/or raw plasma based at least in part on the donor's extracellular fluid amount, to control the system to operate draw and return phases to withdraw whole blood from the donor and separate the whole blood into the plasma product and the second blood component and to return the second blood component to the donor, wherein the control circuit is programmed to perform the draw and return phases at least three times and the control circuit is programmed to perform a final draw phase by drawing a volume of whole blood which is less than a volume of whole blood drawn in a prior draw phase.

A thirty-seventh embodiment includes the system of the thirty-sixth embodiment wherein the control circuit is programmed to receive donor height, sex and hematocrit (Hct) over a network from a donor management system used for qualification screening,

A thirty-eighth embodiment includes the system of the thirty-sixth embodiment, wherein the control circuit is further configured to operate the draw and return phases until a volume of raw plasma (VRP) in the collection container equals the target volume of raw plasma, the volume of raw plasma (VRP) based on a measured volume of plasma product (VPP).

A thirty-ninth embodiment includes the system of the thirty-sixth embodiment, wherein the control circuit is configured to calculate the extracellular fluid amount of the donor as an extracellular fluid volume (ECFV) based on the weight (BM) and height (H) of the donor such that ECFV (L)=0.02154×BM^{0.6469} (kg)×H^{0.7236} (cm).

A fortieth embodiment includes the system of the thirty-sixth embodiment, wherein the control circuit is configured to calculate the extracellular fluid amount of the donor as an extracellular fluid volume (ECFV) based on the weight (BM) and height (H) of the donor such that ECFV (L)=sqrt(BM (kg))×H (m).

In a forty-first embodiment, a method for collecting plasma in which plasma product is collected in multiple collection phases between which separated red blood cells are reinfused to the donor is provided. The method of this embodiment includes a) determining a volume of whole blood (V_{b}) and hematocrit (Hct) for a donor; b) determining a volume of raw plasma (V_{RP}) that may be collected from the donor; c) determining a volume of plasma product (V_{PP}) that may be collected, wherein the plasma product comprises the raw plasma volume plus a volume of anticoagulant; d) withdrawing whole blood from the donor; e) introducing anticoagulant into the withdrawn whole blood at a specified ratio (ACR); f) separating the withdrawn whole blood into a plasma product and a second component comprising red blood cells; g) collecting the plasma product in a plasma collection container; h) after a desired amount of whole blood has been withdrawn from the donor, returning the red blood cells to the donor; and i) determining the Hct of the donor and V_{PP} prior to each collection phase.

A forty-second embodiment includes the method of the forty-first embodiment wherein steps d)-i) are continued until a measured volume of plasma product in the collection container equals V_{PP}.

In a forty-third embodiment a method for collecting plasma in which plasma product is collected in multiple collection phases between which separated red blood cells are reinfused to the donor is provided. The method comprises: a) determining a volume of whole blood (V_{b}) and hematocrit (Hct) for a donor; b) determining a volume of raw plasma (V_{RP}) that may be collected from the donor based on V_{b}; c) determining a volume of anticoagulant V_{AC} to be added to the V_{RP} based on an anticoagulant ratio (ACR) and the Hct of the donor, such that V_{AC}=V_{RP}*(ACR*(1−Hct)); d) determining a volume of plasma product (V_{PP}) that may be collected, wherein the plasma product comprises the raw plasma volume (V_{RP}) plus the volume of anticoagulant (V_{AC}); e) withdrawing whole blood from the donor; f) introducing anticoagulant into the withdrawn whole blood at the specified ratio (ACR); g) separating the withdrawn whole blood into a plasma product and a second component comprising red blood cells; h) collecting the plasma product in a plasma collection container; i) after a desired amount of whole blood has been withdrawn from the donor, returning the red blood cells to the donor; and j) determining the Hct of the donor and V_{PP} prior to each collection phase.

A forty-fourth embodiment includes the method of forty-third embodiment wherein steps d)-j) are continued until a measured volume of plasma product in the collection container equals V_{PP}.

A forty-fifth embodiment includes the method of any one of embodiments forty-three and forty-four wherein V_{b} is determined based on one or more donor specific characteristics including a donor's weight, height, sex, age, and morphology.

In a forty-sixth embodiment a method for collecting a volume of plasma product (V_{PP}) in an apheresis procedure in which plasma product is collected in multiple collection phases between which separated red blood cells are reinfused to the donor is provided. In the method, V_{PP} is equal to a volume of raw plasma (V_{RP}) that may be collected from a donor plus a volume of anticoagulant (V_{AC}) that is added to the V_{RP} during the apheresis procedure. The steps of the method comprise: a) determining a weight (W_{kg}) and sex (M or F) for the donor; b) determining a hematocrit (Hct) for the donor; c) determining the volume of raw plasma (V_{RP}) that may be collected based on the weight (W_{kg}) and sex (M or F) of the donor; d) determining a ratio K between the V_{PP} and the V_{RP}, such that K=V_{PP}/V_{RP}, based on an anticoagulant ratio and the Hct of the donor; e) determining V_{PP}, such that V_{PP}=V_{RP}*K; f) withdrawing whole blood from the donor; g) introducing anticoagulant into the withdrawn whole blood at a specified ratio (ACR); h) separating the withdrawn whole blood into a plasma product and a second component comprising red blood cells; i) collecting the plasma product in a plasma collection container; j) after a desired amount of whole blood has been withdrawn from the donor, returning the red blood cells to the donor; and k) determining the Hct of the donor and the target V_{PP} prior to each collection phase.

A forty-seventh embodiment includes the method of embodiment forty-six wherein steps c)-k) are repeated until a measured volume of plasma product in the collection container equals V_{PP}. Preferably, K=V_{PP}/V_{RP}=(ACR*(1−Hct/100)+1)/(ACR*(1−HCT/100)).

In a forty-eighth embodiment a method for collecting a volume of plasma product (V_{PP}) in an apheresis procedure in which plasma product is collected in multiple collection phases between which separated red blood cells are reinfused to the donor is provided. In this method, V_{PP} is equal to a volume of raw plasma (V_{RP}) that may be collected from a donor plus a volume of anticoagulant (V_{AC}) that is added to the V_{RP} during the apheresis procedure. The steps of the method comprise: a) determining a weight (W_{kg}) and sex (M or F) for the donor; b) determining a hematocrit (Hct) for the donor; c) determining the volume of raw plasma (V_{RP}) that may be collected based on the weight of the donor (W_{kg}) and the sex (M or F) of the donor; d) determining the V_{AC} to be added to the V_{RP} based on an anticoagulant ratio (ACR) and the Hct of the donor, such that V_{AC}=V_{RP}*(ACR*(1−Hct)); e) determining V_{PP}, such that V_{PP}=V_{RP}+V_{AC}; f) withdrawing whole blood from the donor; g) introducing anticoagulant into the withdrawn whole blood at a specified ratio (ACR); h) separating the withdrawn whole blood into a plasma product and a second component comprising red blood cells; i) collecting the plasma product in a plasma collection container; j) after a desired amount of whole blood has been withdrawn from the donor, returning the red blood cells to the donor; and k) determining the Hct of the donor and V_{PP} prior to each collection phase.

A forty-ninth embodiment includes the method of embodiment forty-eight wherein steps d)-k) are continued until a measured volume of plasma product in the collection container equals V_{PP}.

A fiftieth embodiment includes the method of any one of embodiments forty-eight and forty-nine wherein V_{RP} is determined by establishing the V_{RP} for each of a plurality of ranges of donor weight and selecting the V_{RP} for the range of weight that is inclusive of the weight of the donor. The ranges of donor weight may be in three categories from 110 to 149 lbs., 150 to 174 lbs., and 175 lbs. and up.

In a fifty-first embodiment V_{RP}=K₁*W_{kg}.

In a fifty-second embodiment V_{RP} is no greater than 28.6% of (1−Hct)*(V_{b}).

In a fifty-third embodiment V_{b} is determined using one of Nadler's equations, Gilcher's Rule of Five, the standards of the ICSH, and any other generally accepted methodology.

In a fifty-fourth embodiment V_{RP}=W_{kg}*10 ml/kg.

In a fifty-fifth embodiment wherein donor parameters are used to estimate a total blood volume (V_{b}) for the donor, V_{RP}=K₂*V_{b}.

In a fifty-sixth embodiment, an automated system for separating plasma from whole blood is provided comprising a reusable hardware component and a disposable kit is provided. The disposable kit further comprises i) a separator for separating whole blood into a plasma fraction and a concentrated cell fraction, the separator having an input having a blood line integrally connected thereto for transporting whole blood from a donor to the separator, a plasma output port integrally connected to a plasma collection container by a plasma line, and a concentrated cell outlet port integrally connected to a reservoir for receipt of concentrated cells prior to reinfusion to the donor; ii) an anticoagulant line integrally connected to the blood line and configured to be connected to a source of anticoagulant for transporting anticoagulant to the donor line, iii) a saline line configured to be attached to a source of saline for transporting saline to the blood line, and iv) a reinfusion line for transporting concentrated cells from the reservoir to the donor line. The reusable hardware component further comprises i) a first peristaltic pump for delivering anticoagulant at a controlled rate into the blood line during a collection phase, ii) a second pump for delivering anticoagulated whole blood to the separator during the collection phase and for returning concentrated cellular components during a reinfusion phase, iii) a third pump for delivering concentrated cellular components from the separator to the reservoir during the collection phase, iv) a clamp associated with each of the blood line, plasma line, reinfusion line and saline line, v) a weigh scale for weighing each of the plasma collection container, the reservoir and the source of anticoagulant, and vi) a programmable controller comprising a touch screen for receiving input from an operator, the programmable controller configured to receive a signal from each of the weigh scales and to automatically operate the first, second and third pumps and the clamps to separate whole blood into a plasma fraction and a concentrated cell fraction during the collection phase and to return concentrated cells to the donor during the reinfusion stage. The programmable controller is further configured to determine the weight of the plasma fraction to be collected in the plasma collection container in accordance with any of the aspects described herein, and to terminate the collection phase upon receiving a signal from the weigh scale for the plasma collection container equal to the weight of the plasma fraction determined by the controller. In determining the target amount for the plasma product to be collected, the controller may be configured to calculate the hematocrit of the donor prior to the collection phase of each cycle. Alternatively, or additionally, the controller may receive a signal from a sensor or the like that is indicative of the donor's hematocrit. Further, the amount of plasma product in the plasma collection container may be determined by, e.g., the weigh scale associated with the plasma collection. In one embodiment, the separator comprises a spinning membrane separator.

While the present application describes calculating target raw plasma and/or plasma product based on total blood volume, total plasma volume, extracellular fluid volume, and/or other parameters, in alternative embodiments, target raw plasma and/or plasma product may be calculated based on one or more other donor parameters, such as intracellular fluid amount, interstitial fluid amount, tissue fluid amount, intravascular fluid amount, cerebrospinal fluid amount, total body water, lymph amount, transcellular fluid, effective circulating volume, or other donor parameters. Target raw plasma and/or plasma product may further be calculated or determined based at least in part on electrolytic constituents, such as cations and anions, such as sodium, potassium, calcium, chloride, bicarbonate, and/or phosphate.

It will be understood that the embodiments described are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope of the claims is not limited to the above description but is set forth in the following claims.

1. A system for collecting plasma, comprising:
a separator configured to separate the whole blood into a plasma product and a second blood component comprising concentrated cells, the blood separator having a plasma output port coupled to a plasma line configured to send the plasma product to a plasma product collection container;
a donor line configured to introduce the whole blood from the donor to the separator, flow through the donor line being controlled by a first pump;
an anticoagulant line coupled to an anticoagulant source, flow through the anticoagulant line being controlled by a second pump to combine anticoagulant with the whole blood from the donor based on an anticoagulant ratio;
an input device configured to receive input from an operator and/or over a network from a remote computer; and
a control circuit configured to control operation of the system, the control circuit coupled to the input device and configured to receive one or more donor parameters, to estimate a physiological fluid amount of the donor based at least in part on the one or more donor parameters, to calculate a target amount of plasma product comprising raw plasma and anticoagulant by multiplying a prestored constant by the estimated physiological fluid amount, the control circuit configured to control the system to operate draw and return phases to withdraw whole blood from a donor and separate the whole blood into the plasma product and the second blood component and to return the second blood component to the donor, wherein the control circuit is further configured to operate the draw and return phases until a measured amount of plasma product in the collection container meets the target amount of plasma product.

2. The system of claim 1, wherein the control circuit is configured to not use donor hematocrit in determining when the measured amount of plasma product meets the target amount of plasma product.

3. The system of claim 1, wherein the control circuit is configured to not use donor hematocrit in calculating the target amount of plasma product.

4. The system of claim 1, wherein the estimated physiological fluid amount is an extracellular fluid volume.

5. The system of claim 4, wherein the prestored constant is between about 0.050 and about 0.064.

6. The system of claim 4, wherein the control circuit is configured to calculate the extracellular fluid volume of the donor based on the weight (W), height (H), and sex of the donor such that ECFV=0.0399×W^{0.6065}×H^{0.6217} for females and ECFV=0.0755×W^{0.6185}×H^{0.4982} for males.

7. The system of claim 1, wherein the estimated physiological fluid amount is a total plasma volume.

8. The system of claim 7, wherein the prestored constant is between about 0.25 and about 0.34.

9. The system of claim 1, wherein the estimated physiological fluid amount is a total blood volume.

10. The system of claim 9, wherein the prestored constant is between about 0.10 and about 0.75.

11. The system of claim 1, wherein the prestored constant is different for a male donor than for a female donor, donor gender data being received from the input device and used to select from a male prestored constant and a female prestored constant.

12. The system of claim 1, wherein a weight and a height of the donor are received over a network from a donor management system, wherein the donor management system is used for qualification screening.

13. The system of claim 1, wherein the target amount for plasma product is calculated prior to initiating blood collection from the donor.

14. The system of claim 1, wherein the control circuit is configured to measure the amount of plasma product comprising raw plasma and anticoagulant in the collection container using a weigh scale.

15. A method for collecting plasma comprising:
(a) receiving one or more donor parameters selected from donor weight, donor height, donor body mass index and donor sex;
(b) estimating a donor physiological fluid amount based, at least in part, on the one or more donor parameters, wherein the estimated donor physiological fluid amount is selected from total blood volume, total plasma volume and extracellular fluid volume;
(c) calculating a target amount of plasma product to collect, the target amount of plasma product comprising raw plasma and anticoagulant, the target amount of plasma product calculated by multiplying the estimated donor physiological fluid amount by a prestored constant;
(d) withdrawing whole blood from the donor through a venous-access device to a blood component separation device;
(e) introducing anticoagulant into the withdrawn whole blood;
(f) separating the withdrawn whole blood into a plasma product comprising raw plasma and anticoagulant and at least a second blood component;
(g) collecting the plasma product from the blood component separation device into a plasma collection container;
(h) determining whether the collected plasma product comprising raw plasma and anticoagulant has reached the target amount of plasma product to collect; and
(i) continuing steps (d) through (h) until the collected plasma product reaches the target amount of plasma product to collect.

16. The method of claim 15, wherein the estimated donor physiological fluid amount is donor extracellular fluid.

17. The method of claim 16, wherein the prestored constant is between about 0.05 and about 0.07.

18. The method of claim 17, wherein the prestored constant comprises a first constant for use with a male donor and a second constant different than the first constant for use with a female donor.

19. The method of claim 15, wherein actual donor hematocrit is not used in estimating donor physiological fluid amount, wherein actual donor hematocrit is not used in calculating a target amount of plasma product to collect.

20. A system for collecting plasma, comprising:
a blood separator configured to separate the whole blood into a plasma product and a second blood component comprising concentrated cells, the blood separator having a plasma output port coupled to a plasma line configured to send the plasma product to a plasma product collection container, the plasma product comprising raw plasma and anticoagulant;
a donor line configured to introduce the whole blood from the donor to the blood separator, flow through the donor line being controlled by a first pump;
an anticoagulant line coupled to an anticoagulant source, flow through the anticoagulant line being controlled by a second pump to combine anticoagulant with the whole blood from the donor based on an anticoagulant ratio;
an input device configured to receive input from an operator; and
a control circuit programmed to control operation of the system, the control circuit coupled to the input device and programmed to receive at least a donor's weight, to estimate a donor physiological fluid amount based at least in part on the donor's weight and without using the donor's actual hematocrit, to determine a target volume for plasma product comprising raw plasma and anticoagulant as a predetermined percentage of the donor's physiological fluid amount and without using the donor's actual hematocrit, to control the system to operate draw and return phases to withdraw whole blood from the donor and separate the whole blood into a the collected plasma product and the second blood component and to return the second blood component to the donor, wherein the control circuit is programmed to perform the draw and return phases at least three times, wherein the control circuit is configured to compare the amount of collected plasma product to the target volume of plasma product and to continue processing the whole blood until the amount of collected plasma product reaches the target volume of plasma product.
